(19) United States
(12) Patent Application Publication
(10) Pub. No.: US 2020/0121620 A1
(43) Pub. Date: Apr. 23, 2020

(54) SENOLYTIC COMPOUNDS

(71) Applicant: UNITED KINGDOM RESEARCH AND INNOVATION, Swindon (GB)

(72) Inventors: Jesus GIL, London (GB); Ana GUERRERO, London (GB); Nicolas HARRANZ, London (GB)

(21) Appl. No.: 16/616,251

(22) PCT Filed: May 25, 2018
(86) PCT No.: PCT/GB2018/051437

§ 371 Date: Nov. 22, 2019

(30) Foreign Application Priority Data
May 26, 2017 (GB) 1708456.7

(51) Int. Cl. A61K 31/137; A61K 31/18; A61K 31/34; A61K 31/03; A61K 31/352; A61K 31/122; A61K 31/4745; A61K 31/704; A61K 31/4709; A61K 31/475; A61K 31/426; A61K 38/13

(52) U.S. Cl. CPC A61K 31/137; A61K 31/18; A61K 31/34; A61K 31/03; A61K 31/352; A61K 31/122; A61K 45/06; A61K 31/704; A61K 31/4709; A61K 31/475; A61K 31/426; A61K 38/13; A61K 31/4745

(57) ABSTRACT

The present invention relates to an agent for use in selectively killing one or more senescent cells, wherein said agent is selected from the following: a cardiac glycoside or alglycone, a focal adhesion kinase (FAK) inhibitor, an HMG-CoA reductase inhibitor, JFD00244, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Nitazoxanide, Nitrofurazone, Temsirolimus, Eltrombopag, Adapalene, Azacyclonol, Enoxacin and Raltegravir, and pharmaceutically acceptable salts thereof.

Another aspect relates to compounds for use in treating or preventing a senescence-associated disease or disorder, and methods relating thereto.

The present invention relates to compounds that are capable of selectively killing senescent cells over non-senescent cells, i.e. senolytic compounds. The present invention also provides compounds for use in treating senescence-associated diseases or disorders, and methods relating thereto.

### CROSS-REFERENCE TO RELATED APPLICATIONS

This application is a national phase entry under 35 USC § 371 of International Application No. PCT/GB2018/051437, filed May 25, 2018, which claims priority to Great Britain Patent Application No. 1708456.7, filed May 26, 2017. The entire contents of these applications are incorporated herein by reference in their entireties

### BACKGROUND TO THE INVENTION

Senescence is a cellular programme that imposes a stable arrest on damaged or old cells to avoid their replication. As well as growth arrest, senescent cells undergo profound phenotypic changes that include chromatin reorganisation, increase of 3-galactosidase activity (referred to as senescence-associated β-galactosidase or SA-β-Gal) and secretion of multiple factors, mainly pro-inflammatory, that are collectively referred to as the senescence-associated secretory phenotype (SASP).

Replicative senescence is activated upon serial passage of cells in culture (or as cells become older in an organism). Senescence can also be induced by range of different insults that include oncogene activation, irradiation and exposure to chemotherapeutic drugs. In addition, there are several drugs, the prototypic example being CDK4/CDK6 inhibitors such as Palbociclib, which induce senescence.

Mechanisms of Senescence

The stable growth arrest characteristic of senescence is implemented by the activation of the p16/Rb and p53/p21 pathways. The cyclin-dependent kinase inhibitors p16^{INK4a} and p21Cip1 inhibit CDK activity, resulting in Rb hypophosphorylation and G1 growth arrest (Kuilman et al., 2010). Moreover, p16^{INK4a} is specifically induced during senescence and used to identify senescent cells alone or in combination with other markers such as SA-β-Gal activity, formation of senescence-associated heterochromatin foci (SAHF) and others.

### Senescence and Disease

Senescent cells accumulate during age and are associated with many diseases, including cancer, fibrosis and many age-related pathologies. Recent evidence suggests that senescent cells are detrimental in multiple pathologies and their elimination confers many advantages, ameliorating multiple pathologies and increasing healthspan and lifespan.

Senescent cells are present in many pre-neoplastic lesions, fibrotic tissues (e.g. in the liver, kidney, heart, pancreas) and old tissues. Senescent cells are also associated with a long list of other pathologies (Munoz-Espin and Serrano, 2014), including neurological (e.g. brain aneurysm, Alzheimer's and Parkinson), pulmonary (e.g. idiopathic pulmonary fibrosis, chronic obstructive pulmonary disease and cystic fibrosis), ophthalmological (e.g. cataracts, glaucoma, macular degeneration), musculoskeletal (e.g. sarcopenia, disc degeneration, osteoarthritis), cardiovascular (e.g. atherosclerosis, cardiac fibrosis, aorta aneurysm), renal (e.g. kidney disease, transplant complications) and others such as diabetes, mucositis, hypertension and osteomyelofibrosis (OMF).

While senescent cells have a protective role against cancer and limit most types of fibrosis, the accumulation of senescent cells during ageing and many other diseases is thought to be detrimental. To date, there is no unequivocal explanation of why senescent cells are detrimental.

### Benefits of Eliminating Senescent Cells

Evidence for the many detrimental effects of senescent cells (and the benefits caused by their selective elimination) was provided by a series of studies from the van Deursen lab (Baker et al., 2016; Baker et al., 2011; Childs et al., 2016). These studies used transgenic mice (INK4-ATTAC mice) that express an inducible fusion protein specifically on senescent cells (taking advantage of the promoter of p16^{Ink4a}). Activation of this fusion protein, by addition of a drug that triggers its dimerization, results in the selective death of senescent cells.

By using this mouse model, it was shown that the clearance of senescent cells increases lifespan and healthspan by attenuating multiple age-related pathologies (Baker et al., 2016; Baker et al., 2011; Childs et al., 2016). Clearance of senescent cells delayed tumorigenesis, and attenuated cataract formation, atherosclerosis and the age-related deterioration of kidney, fat and heart amongst other organs. The results obtained with INK4-ATTAC mice have been replicated in part in a different mouse model in which a tk transgene is expressed in senescent cells (3MR mice), allowing for their selective elimination upon gancyclovir treatment (Demaria et al., 2014). Moreover, the elimination of senescent cells in chemotherapy reduced cancer recurrence and the side effects associated with chemotherapy (Demaria et al., 2016). Importantly, the elimination of senescent cells has no side effects besides delaying wound healing if the senescent cells are eliminated during the healing process (Baker et al., 2016; Demaria et al., 2014). However, the unifying prevalent hypothesis is that (pro-inflammatory) factors secreted by senescent cells hamper tissue homeostasis. This suggests that common mechanisms, mediated by senescent cells, could be responsible for the effects of many age-related pathologies.

### Senolytic Drugs

Proof-of-concept studies have led to the identification of compounds that can selectively eliminate senescent cells (so-called “senolytics”). Several senolytic compounds have been identified to date, including dasatinib and quercetin (Zhu et al., 2015), piperlongumine (Wang et al., 2016) and Bcl2-family inhibitors such as ABT263 and ABT737 (Chang et al., 2016; Yosef et al., 2016; Zhu et al., 2016). Currently BCl2 family inhibitors are the most promising senolytics, having been shown to kill a range of senescent cells in vivo, with reproducible effects in transgenic mouse models (Chang et al., 2016; Childs et al., 2016; Yosef et al., 2016; Zhu et al., 2016). Bcl2 inhibitors were initially developed as therapies for lymphoma. ABT-737 is a small molecule inhibitor of BCL-2, BCL-XL and BCL-w but has low solubility and oral bioavailability. ABT-263 inhibits the same molecules and is better suited for use in vivo but causes significant thrombocytopenia as a side-effect. Due to the significant side-effects exhibited by senolytic compounds identified to date (including severe neutropenia), there is a need to identify further compounds, and classes of compounds, with senolytic properties.

### STATEMENT OF INVENTION

A first aspect of the invention relates to an agent for use in selectively killing one or more senescent cells, wherein said agent is selected from the following: a cardiac glycoside or aglycone, a focal adhesion kinase (FAK) inhibitor, an HMG-CoA reductase inhibitor, JFD00244, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Nitazoxanide, Nitrofurazone, Temsirolimus, Eltrombopag, Adapalene, Azacyclonol, Enoxacin and Raltegravir, and pharmaceutically acceptable salts thereof.

A second aspect of the invention relates to a method of selectively killing one or more senescent cells in a subject, said method comprising administering to the subject a therapeutically effective amount of an agent selected from the following: a cardiac glycoside or aglycone, a focal adhesion kinase (FAK) inhibitor, an HMG-CoA reductase inhibitor, JFD00244, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Nitazoxanide, Nitrofurazone, Temsirolimus, Eltrombopag, Adapalene, Azacyclonol, Enoxacin and Raltegravir, and pharmaceutically acceptable salts thereof.

A third aspect of the invention relates to a compound for use in treating or preventing a senescence-associated disease or disorder, wherein said compound is an HMG-CoA reductase inhibitor, and said senescence-associated disease or disorder is other than a cardiovascular disorder.

A fourth aspect of the invention relates to a compound for use in treating or preventing a senescence-associated disease or disorder, wherein said compound is a focal adhesion kinase (FAK) inhibitor.

A fifth aspect of the invention relates to a compound for use in treating or preventing a senescence-associated disease or disorder, wherein said compound is selected from the following: JFD00244, Cyclosporine, Tyrphostin AG879, Cantharidin, Diphenyleneiodonium chloride, 2,3-Dimethoxy-1,4-naphthoquinone, LY-367,265, Rotenone, Idarubicin, Dequalinium chloride, Vincristine, Atorvastatin calcium, Nitazoxanide, Nitrofurazone, Temsirolimus, Eltrombopag, Adapalene, Azacyclonol, Enoxacin and Raltegravir, and pharmaceutically acceptable salts thereof, where the compound is not already in salt form.

A sixth aspect of the invention relates to a compound for use in treating or preventing a senescence-associated disease or disorder, wherein said senescence-associated disease or disorder is other than a cardiovascular disorder, and wherein said compound is selected from the following: PF-573228, JFD00244, Ouabain, Bufalin, Digoxin, K-Strophanthin, Strophanthidin, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin, and Raltegravir, and pharmaceutically acceptable salts thereof, where the compound is not already in salt form.

A seventh aspect of the invention relates to a method for treating or preventing a senescence-associated disease or disorder other than a cardiovascular disorder, said method comprising administering to a subject a therapeutically effective amount of an HMG-CoA reductase inhibitor.

An eighth aspect of the invention relates to method for treating or preventing a senescence-associated disease or disorder, said method comprising administering to a subject a therapeutically effective amount of a focal adhesion kinase (FAK) inhibitor.

A ninth aspect of the invention relates to a method for treating or preventing a senescence-associated disease or disorder by selectively killing senescent cells, said method comprising administering to a subject a therapeutically effective amount of a compound selected from the following: PF-573228, JFD00244, Ouabain, Bufalin, Digoxin, K-Strophanthin, Strophanthidin, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin, Raltegravir, NSC 677249 and Defactinib, and pharmaceutically acceptable salts thereof, where the compound is not already in salt form.

A tenth aspect of the invention relates to the use of a compound selected from the following: PF-573228, JFD00244, Ouabain, Bufalin, Digoxin, K-Strophanthin, Strophanthidin, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin, Raltegravir, NSC 677249 and Defactinib, and pharmaceutically acceptable salts thereof, where the compound is not already in salt form;

in the preparation of a medicament for treating or preventing a senescence-associated disease or disorder, wherein said compound selectively kills one or more senescent cells.

An eleventh aspect of the invention relates to the use of a focal adhesion kinase (FAK) inhibitor in the preparation of a medicament for treating or preventing a senescence-associated disease or disorder.

A twelfth aspect of the invention relates to the use of an HMG-CoA reductase inhibitor in the preparation of a medicament for treating or preventing a senescence-associated disease or disorder, wherein said senescence-associated disease or disorder is other than a cardiovascular disorder.

A thirteenth aspect of the invention relates to a senolytic agent which is selected from the following: PF-573228, JFD00244, Ouabain, Bufalin, Digoxin, K-Strophanthin, Strophanthidin, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin, Raltegravir, NSC 677249 and Defactinib, and pharmaceutically acceptable salts thereof, where the compound is not already in salt form.

A fourteenth aspect of the invention relates to compound selected from the following: PF-573228, JFD00244, Ouabain, Bufalin, Digoxin, K-Strophanthin, Strophanthidin, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin, Raltegravir, NSC 677249 and Defactinib, and pharmaceutically acceptable salts thereof, where the compound is not already in salt form; for use in extending the lifespan of a subject, preferably by delaying onset or progression of an age-related disease or condition.

A fifteenth aspect of the invention relates to a compound selected from the following: PF-573228, JFD00244, Ouabain, Bufalin, Digoxin, K-Strophanthin, Strophanthidin, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin and Raltegravir, and pharmaceutically acceptable salts thereof, where the compound is not already in salt form; for use in treating cancer, and wherein said compound:
reduces or eliminates eliminate cancer cells that have been pushed to senescence; or
reduces or eliminates one or more side effects produced by senescent cells, wherein said side effects comprise inflammation, promotion of cancer growth and promotion of metastasis; or
reduces or eliminates one or more side effects of chemotherapy; or
reduces or eliminates one or more side side effects or radiotherapy; or
reduces or eliminates precancerous lesions; or
reduces or eliminates cells that undergo senescence by treatment with CDK4 or CDK6 inhibitors.

The invention is further described with reference to the following figures:

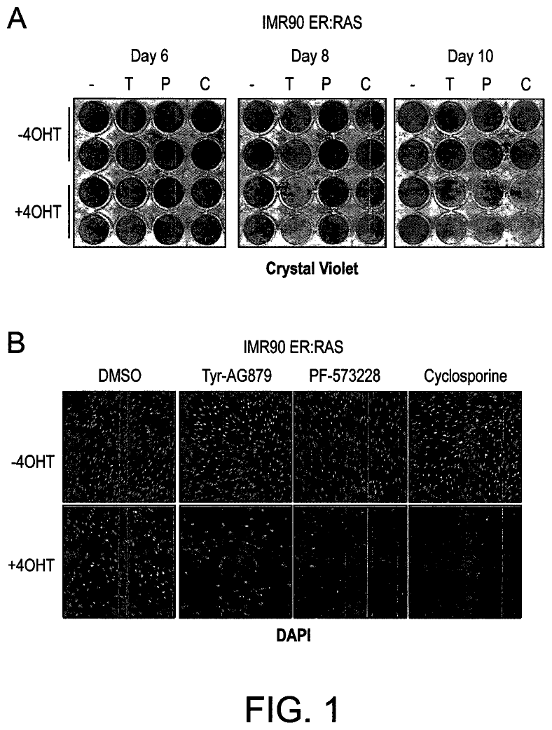

FIG. 1: (A) IMR90 ER:RAS cells were induced to undergo senescence by treating them with 100 nM 4OHT for 6 days. At that point, 10 μM of the indicated drugs was added. In parallel, the same treatments were carried out in IMR90 ER:RAS cells treated with DMSO (−4OHT). These cells do not undergo senescence. Cells were fixed at day 6, 8 and 10 after 4OHT induction and stained with crystal violet to assess cell viability. T, Tyr-AG879; P, PF-573228; C, Cyclosporine. (B) A similar experiment to (A) was conducted and cells at day 10 post-induction were fixed and stained with DAPI to assess cell numbers

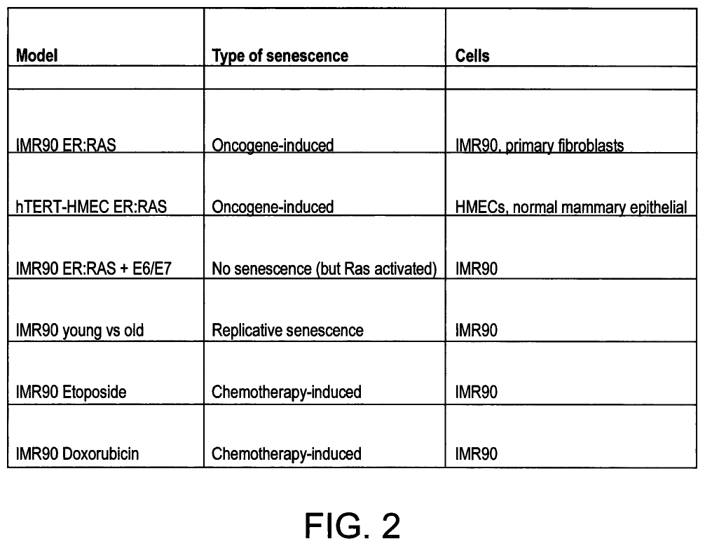

FIG. 2: Summary of the different models of senescence used to test the senolytic compounds in cell culture.

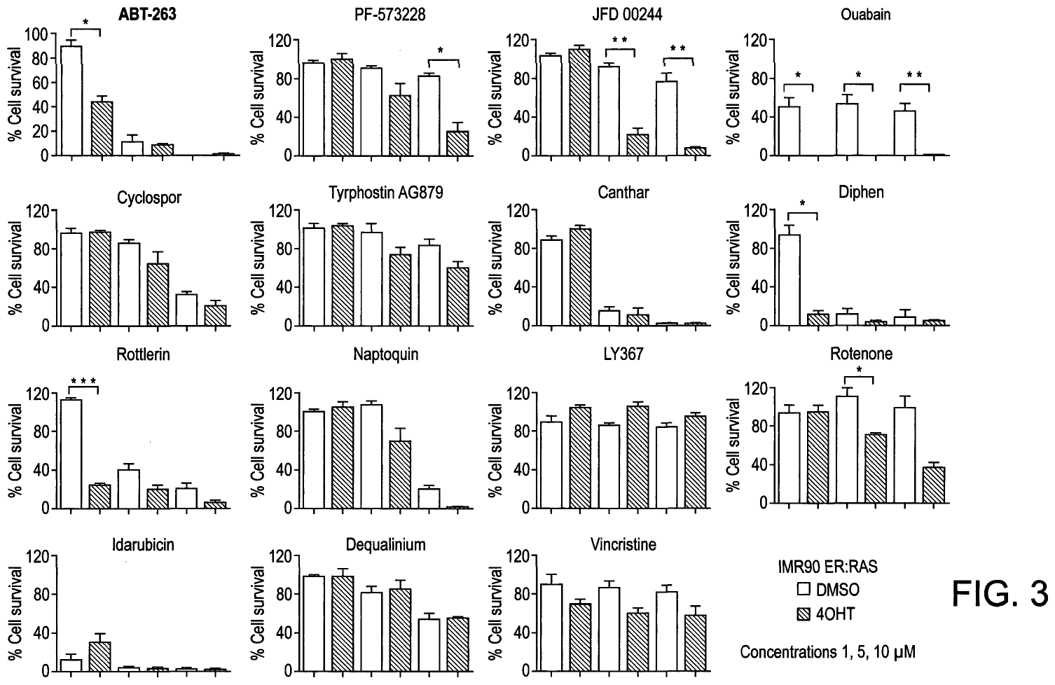

FIG. 3: Testing candidate senolytics for their ability to kill IMR90 cells undergoing oncogene-induced senescence; % cell survival at 1, 5 and 10 μM for DMSO (left bar) and 4OHT (right bar) treatment.

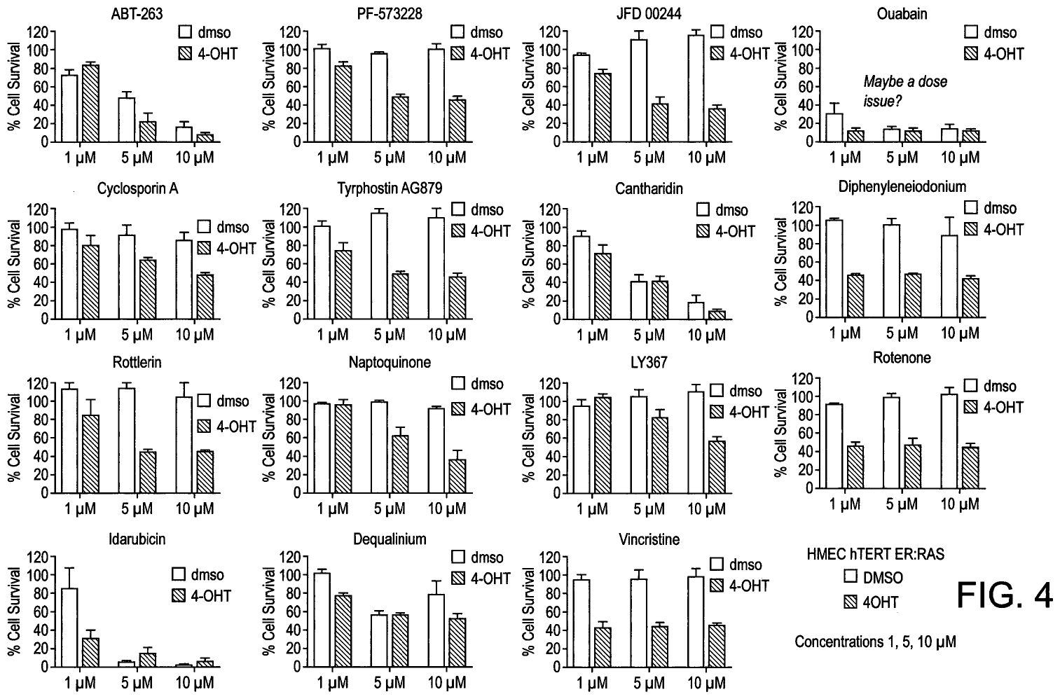

FIG. 4: Testing candidate senolytics for their ability to kill HMEC cells undergoing oncogene-induced senescence; % cell survival at 1, 5 and 10 μM for DMSO (left bar) and 4OHT (right bar) treatment.

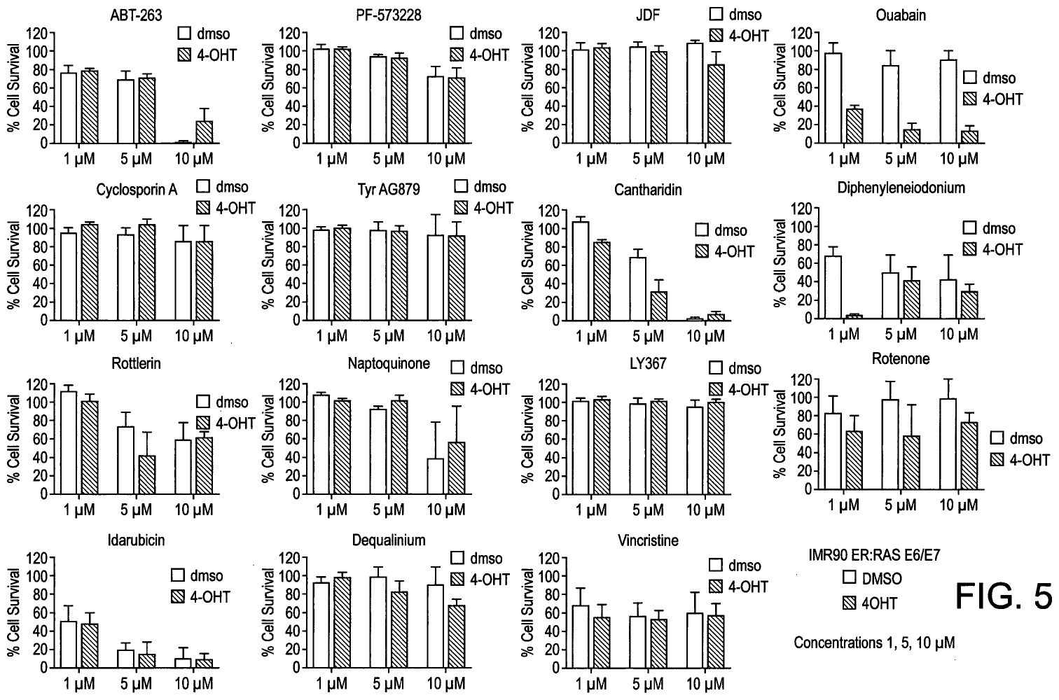

FIG. 5: Testing candidate senolytics for their ability to kill IMR90 ER:RAS E6/E7 cells; % cell survival at 1, 5 and 10 μM for DMSO (left bar) and 4OHT treatment (right bar).

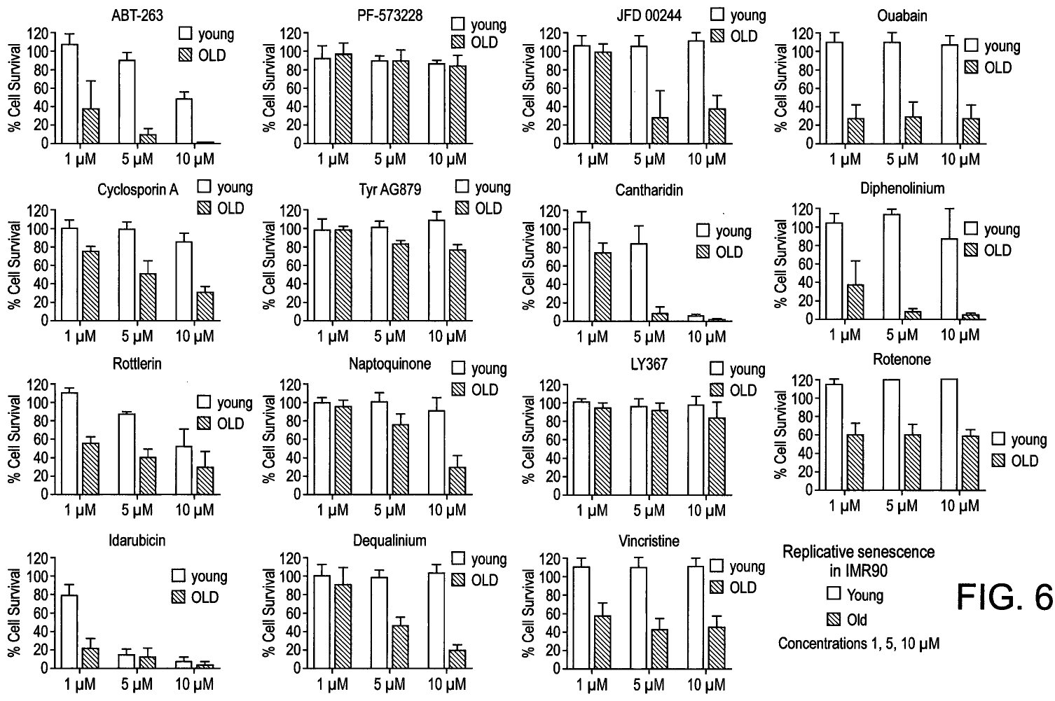

FIG. 6: Testing candidate senolytics for their ability to kill old IMR90 cells; % cell survival at 1, 5 and 10 μM for young (left bar) vs old (right bar).

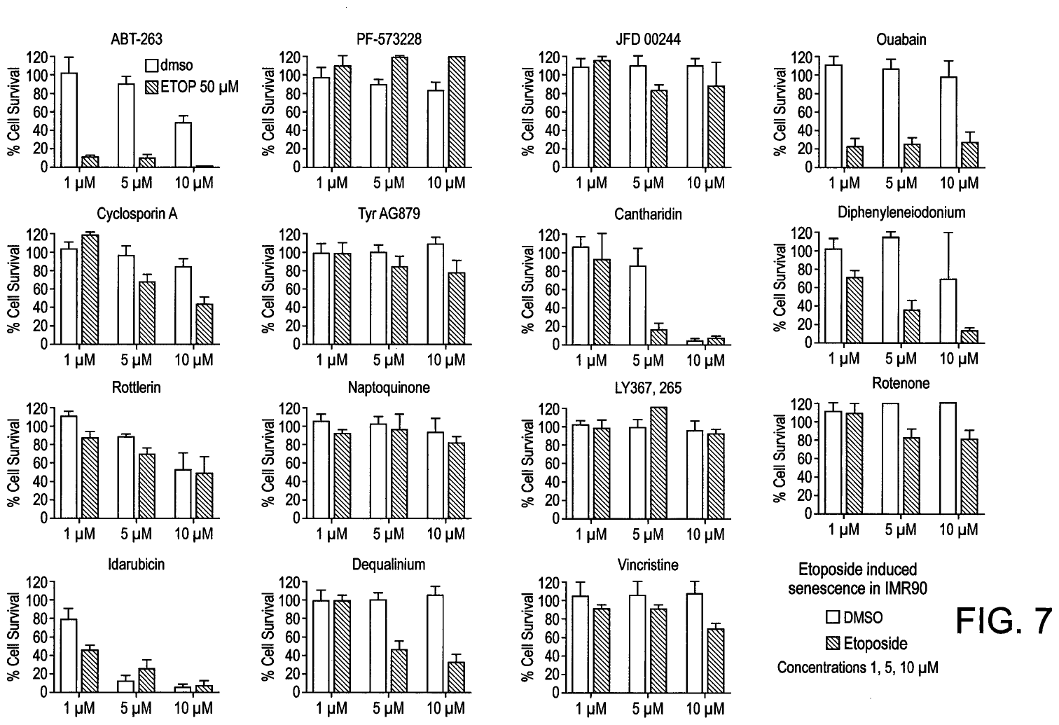

FIG. 7: Testing candidate senolytics for their ability to kill IMR90 cells undergoing senescence by Etoposide treatment; % cell survival at 1, 5 and 10 μM for DMSO (left bar) vs Etoposide (right bar) treatment.

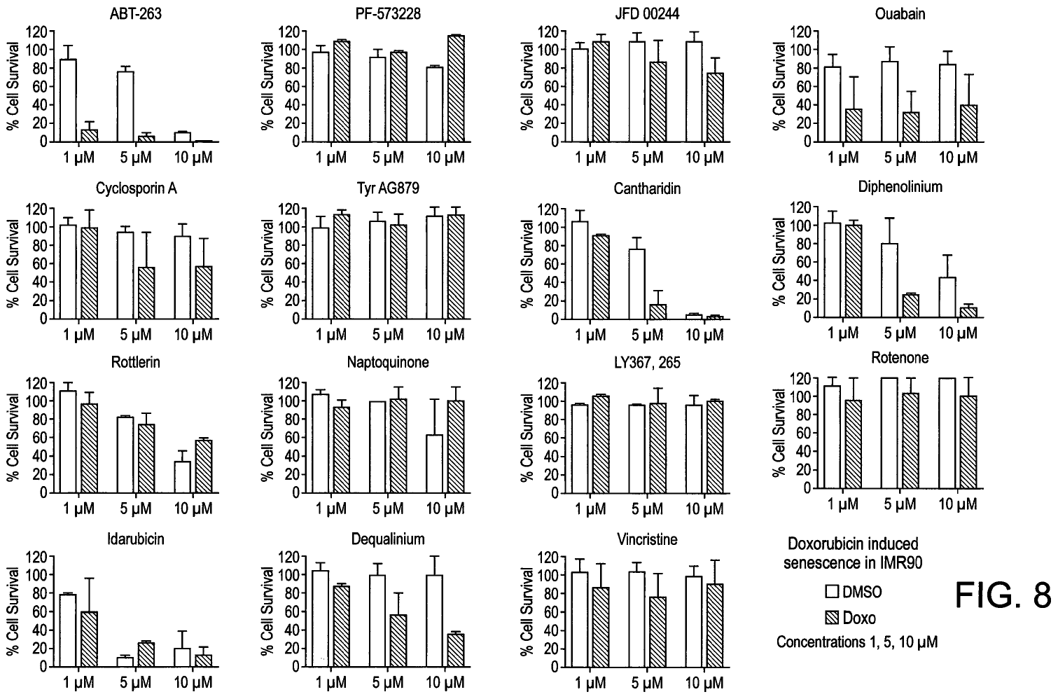

FIG. 8: Testing candidate senolytics for their ability to kill IMR90 cells undergoing senescence by Doxorubicin treatment; % cell survival at 1, 5 and 10 μM for DMSO (left bar) vs (right bar) Doxorubicin treatment.

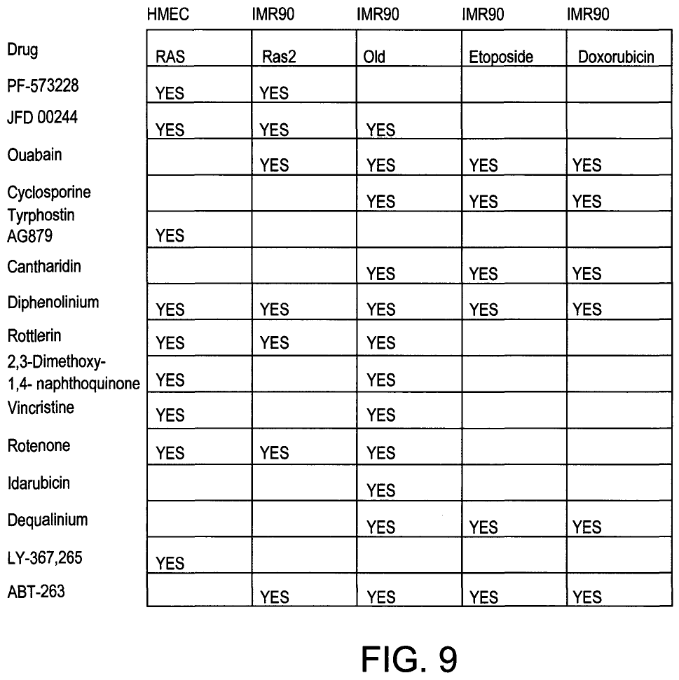

FIG. 9: Summary of the results on testing the candidate senolytics.

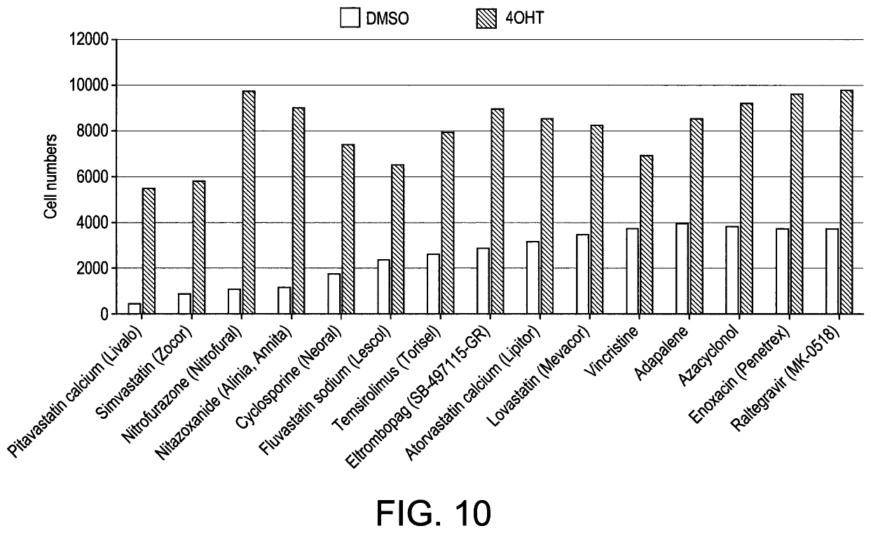

FIG. 10: Testing candidate senolytics for their ability to kill cells undergoing oncogene-induced senescence; cell numbers for DMSO (right bar) and 4OHT (left bar) treatment.

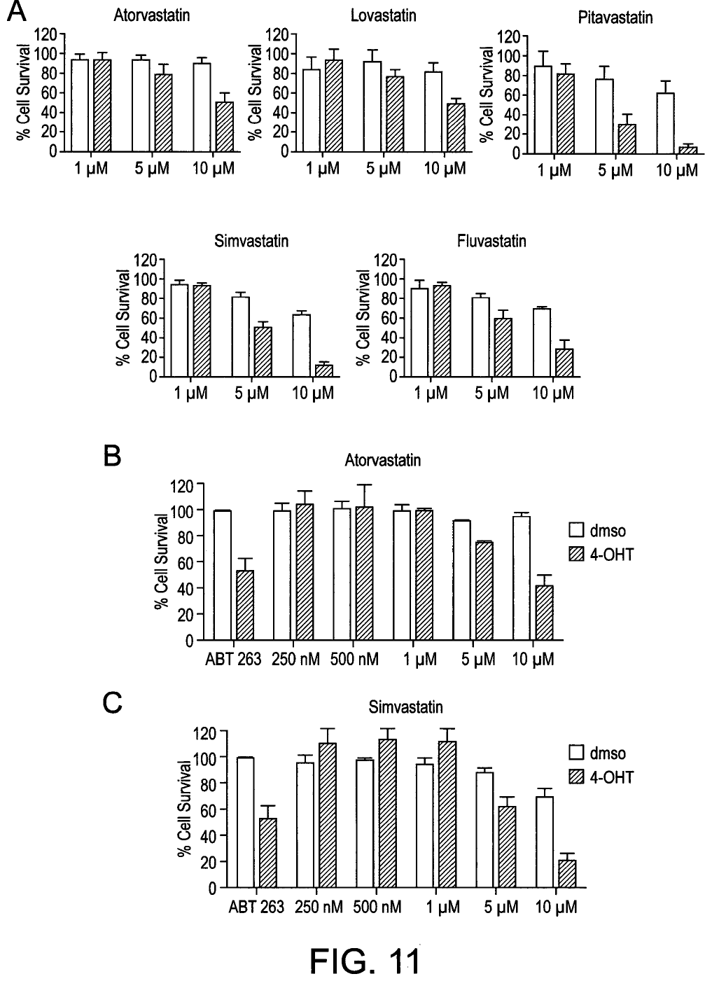

FIG. 11: Statins behave as senolytics, preferentially killing IMR90 ER:RAS cells that undergo oncogene-induced senescence. (A) Testing of statins identified. (B) Atorvastatin behaves as a senolytic in concentration dependent manner. (C) Simvastatin behaves as a senolytic in a concentration dependent manner. For each compound, left bar=DMSO, right bar=4OHT.

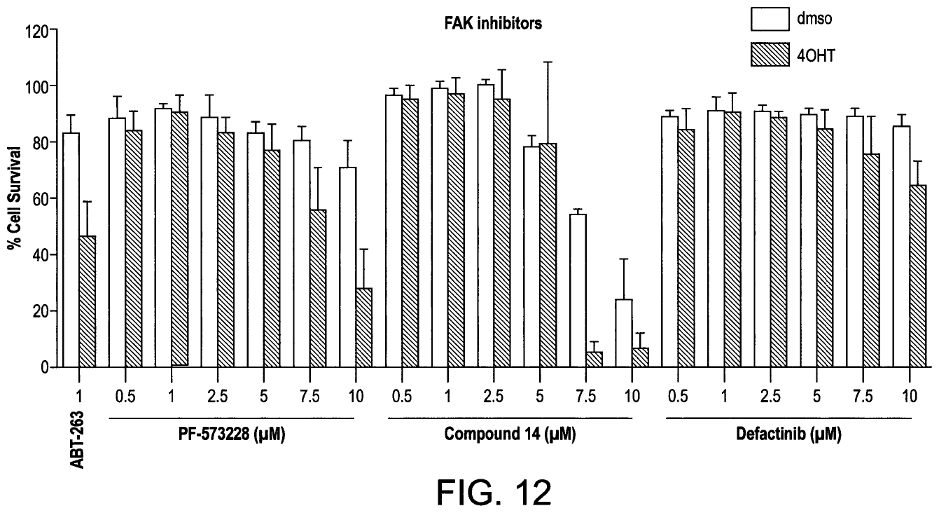

FIG. 12: Different Focal adhesion kinase (FAK) inhibitors behave as senolytics. Three different FAK inhibitors (PF-573228, compound 14 also known as NSC 677249 and Defactinib) were tested on IMR90 ER:RAS cells to analyse their ability to kill differentially senescent cells (4OHT induced; right bar) vs non-senescent cells (DMSO treated; left bar).

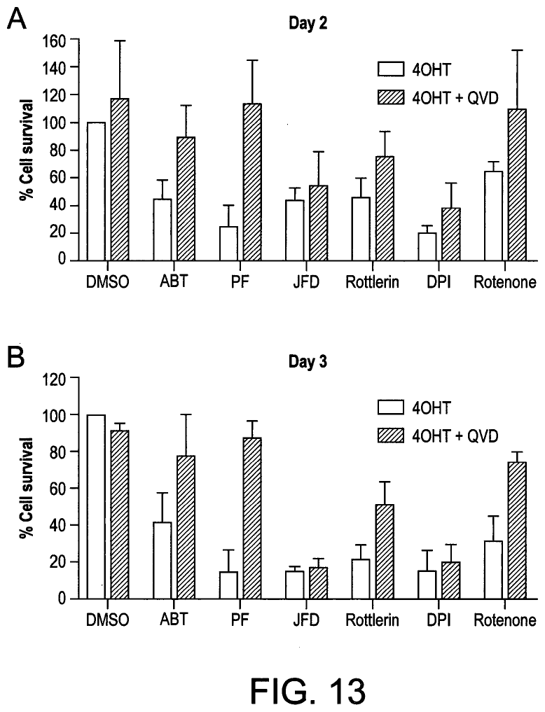

FIG. 13: Several of the senolytic agents kill senescent cells by inducing apoptosis. IMR90 ER:RAS cells were treated with DMSO (as a control) or 4OHT (to induce senescence). Consequently cells were treated with DMSO, ABT-263 or the indicated senolytics for 2 (A) or 3 days (B) in the presence (right bar) or absence (left bar) of the pan-caspase inhibitor QVD. Treatment of senescent cells with senolytics kills them, but is prevented in the case of PF-573228, rottlerin and rotenone by pre-treatment with the pan-caspase inhibitor QVD. ABT, ABT-263; PF, PF-573228; JFD, JFD 00244; DPI, diphenyleneiodonium.

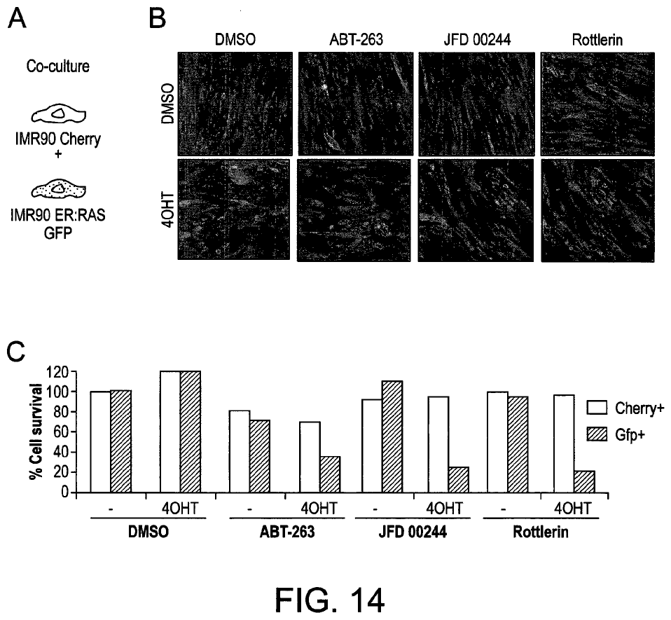

FIG. 14: Senolytic agents JFD 00244 and Rottlerin specifically eliminate senescent cells in mixed cultures containing normal and senescent cells. (A) Setup of the experiments, in which normal cells (IMR90 Cherry) are mixed with IMR90 ER:RAS GFP cells (which become senescent in the presence of 4OHT). (B) Representative pictures showing the mixed cultures treated with the indicated drugs. In the bottom row, upon 4OHT the IMR90 ER:RAS GFP cells are senescent. (C) Quantification of the IMR90 Cherry (left bar) and IMR90 ER:RAS GFP (right bar) in the mixed cultures.

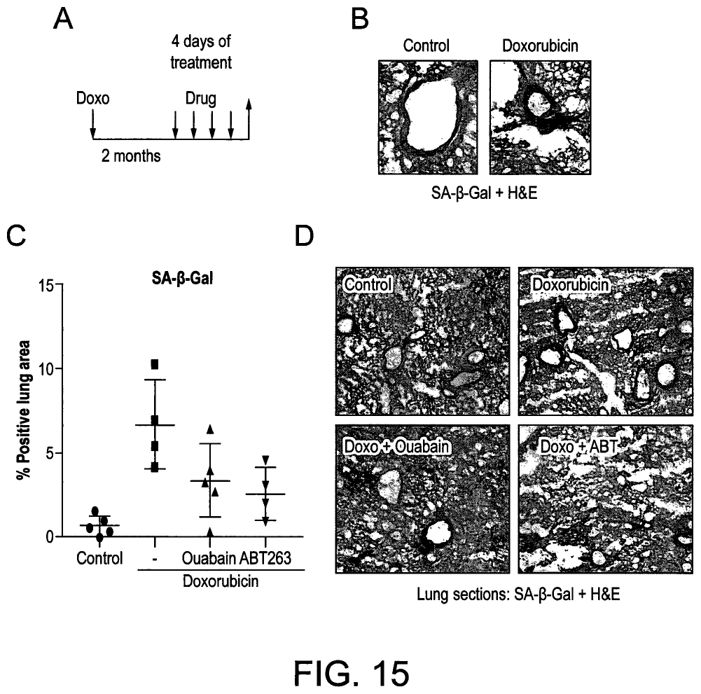

FIG. 15: Ouabain eliminates senescence cells induced by Doxorubicin treatment in mice. (A) Experimental set up, mice (n=5 per group) were administered with a single dose of 10 mg/kg of Doxorubicin. 2 months later mice were treated with vehicle, Ouabain or ABT-263 (as a senolytic control) for 4 consecutive days, before being culled for analysis. (B) Cryosections of lungs obtained from control and doxorubicin treated mice were stained with hematoxilin and eosin (to reveal tissue architecture), and SA-β-Gal (to reveal senescent cells). Senescent cells accumulate in the bronchioles of doxorubicin-treated mice. (C) Quantification of the lung area positive for SA-β-Gal staining. Treatment with doxorubicin induces the accumulation of senescent cells that can be partially eliminated upon treatment with ABT-263 or Ouabain. (D) Representative pictures of lung cryosections.

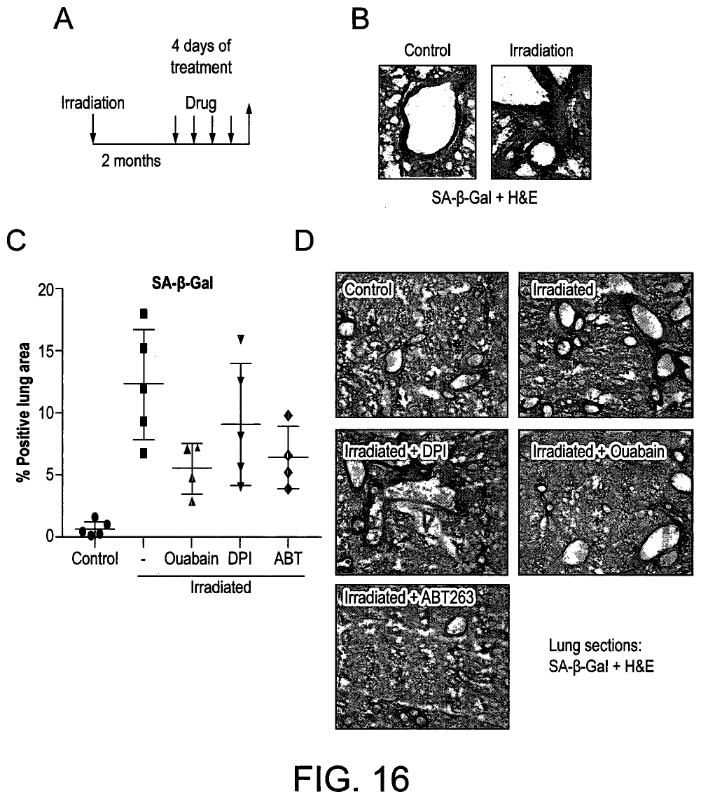

FIG. 16: Treatment with DPI or Ouabain eliminates senescence cells induced by whole body irradiation in mice. (A) Experimental set up, mice (n=5 per group) were irradiated with 6 Gray to induce senescence. 2 months later mice were treated with vehicle, DPI, Ouabain or ABT-263 (as a senolytic control) for 4 consecutive days, before being culled for analysis. (B) Cryosections of lungs obtained from control and irradiated mice were stained with hematoxilin and eosin (to reveal tissue architecture), and SA-β-Gal (to reveal senescent cells). Senescent cells accumulate in the bronchioles of irradiated mice. (C) Quantification of the lung area positive for SA-β-Gal staining. Whole body irradiation induces the accumulation of senescent cells that can be partially eliminated upon treatment with ABT-263, Ouabain or DPI. (D). Representative pictures of lung cryosections.

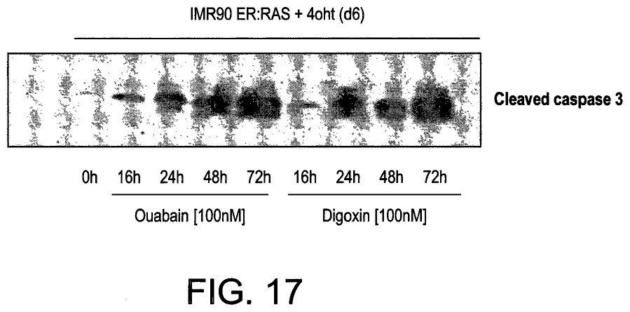

FIG. 17: Treatment with Ouabain or Digoxin induces apoptosis in senescent cells. Western blot showing cleaved caspase 3 upon treatment of IMR90 cells undergoing OIS with cardiac glycosides.

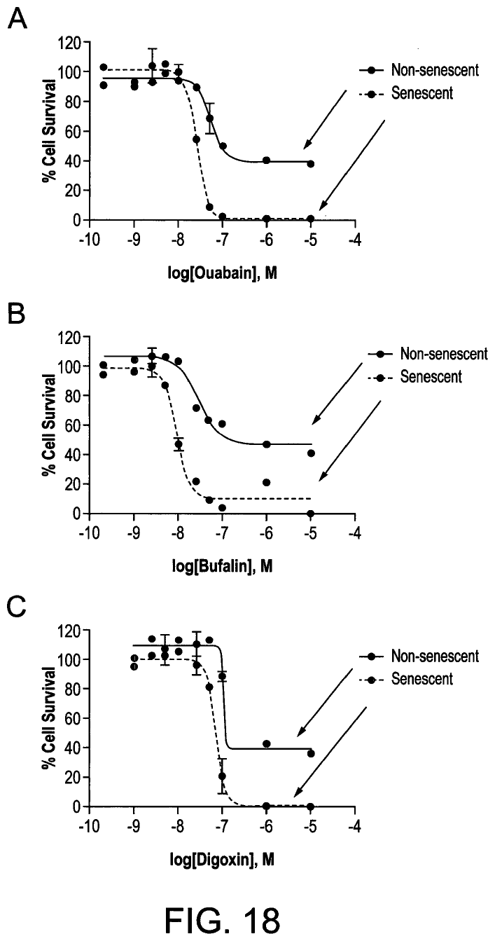

FIG. 18: Cardiac glycosides induce the dose-dependent death of cells undergoing oncogene induced senescence. IMR90 ER:Ras cells were induced to undergo senescence by treatment with 100 nM 4OHT. 6 days after induction cells were treated with (A) Ouabain, (B) Bufalin or (C) Digoxin, at the indicated concentrations.

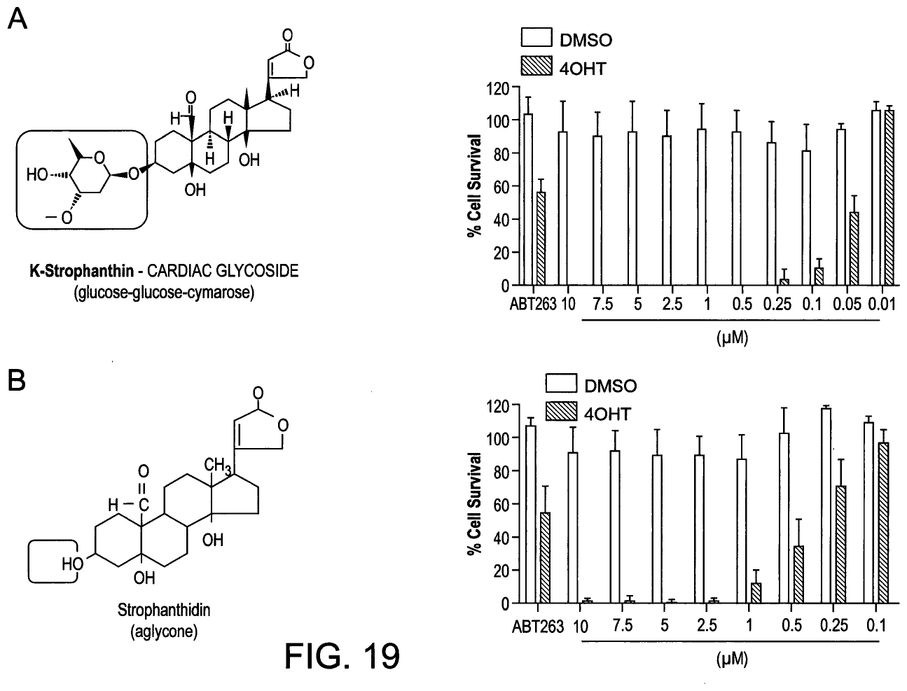

FIG. 19: The glycoside group is not needed for cardiac glycoside-induced senolysis. The ability of a cardiac glycoside (A) to selectively kill senescent cells using the IMR90 ER:RAS system of oncogene-induced senescence was compared to its corresponding cardiac steroid (B, lacking the glycoside group). Left bar=DMSO, right bar=4OHT.

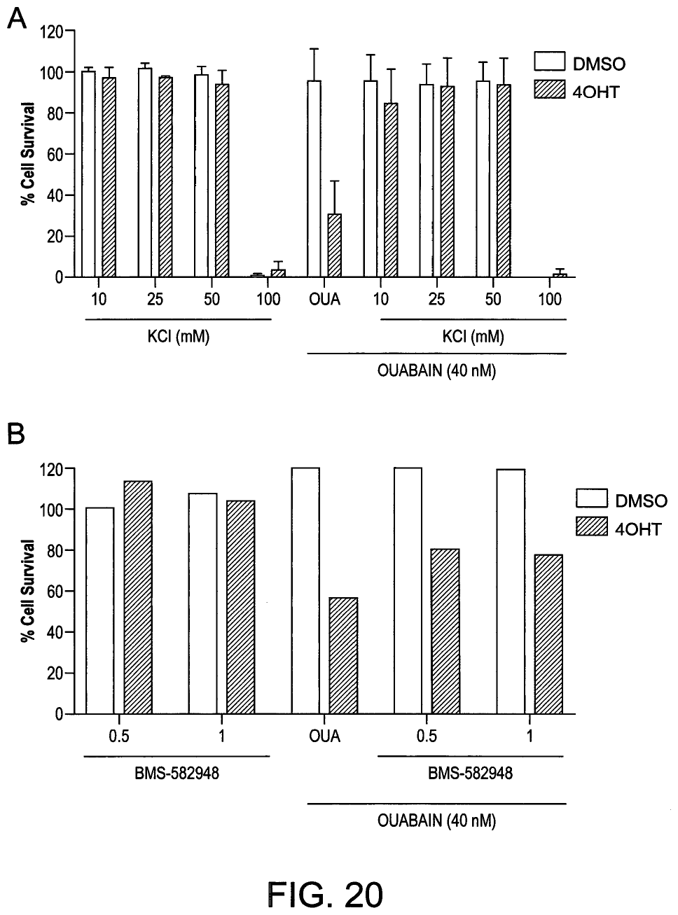

FIG. 20: Senolytic activity of Ouabain is dependent on intracellular K⁺ concentrations and p38. (A) The ability of ouabain to selectively kill IMR90 ER:RAS cells undergoing oncogene-induced senescence was prevented by increasing the intracellular K⁺ concentration by adding KCl, suggesting that it acts on target by inhibiting the K⁺/Na⁺⁺ pump. (B) Inhibition of p38 partially prevents the senolytic properties of ouabain. Left bar=DMSO, right bar=4OHT.

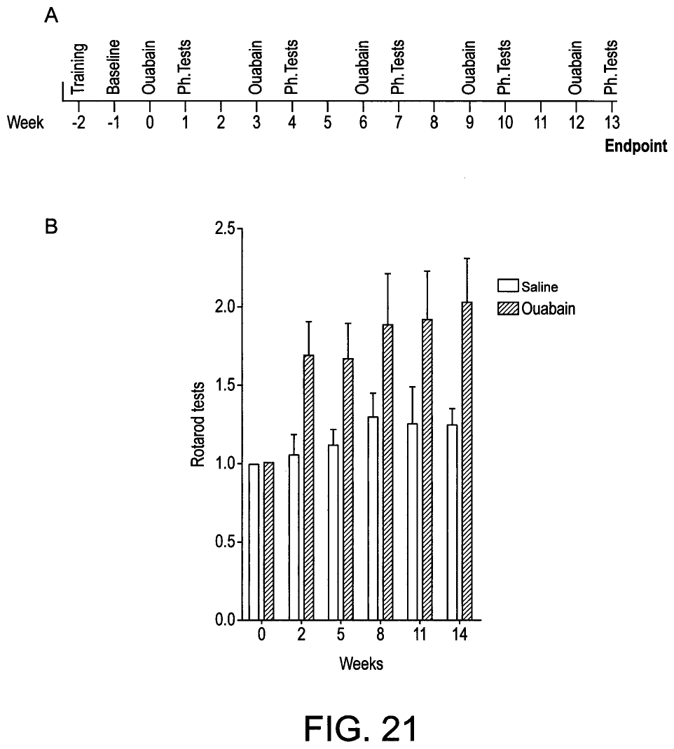

FIG. 21: Intermittent treatment with ouabain improves the physical fitness of old mice. (A) 24 month old female mice were subjected to the schedule summarized in this panel. Ouabain treatment (1 mg/kg ip) was given for 3 consecutive days on treatment weeks. Control group n=7; Ouabain group n=9. (B) Results of rotarod test that assess the physical fitness and coordination of mice. Left bar=saline, right bar=Ouabain.

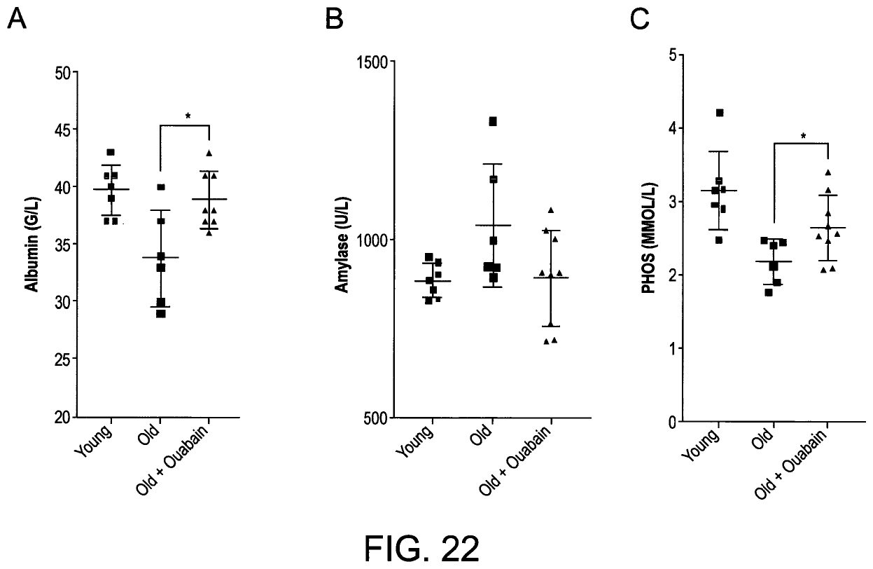

FIG. 22: Ouabain treatment reverts ageing-associated changes of metabolites in blood. The concentrations of Albumin (A), Amylase (B) and PHOS (C) on plasma were measured in mice from the aging experiment described in FIG. 21A. 90 days old female mice were included as a comparison (young).

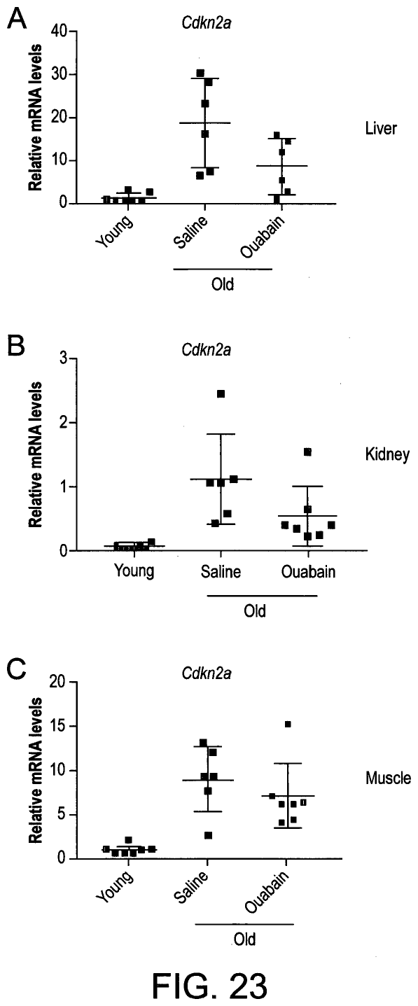

FIG. 23: Old mice treated with Ouabain display reduced senescence in different organs. The expression of Cdkn2a mRNA (encoding for the senescence marker and effector p16^{Ink4a}) increases during aging and can be reduced after ouabain treatment. mRNA levels were measured by qRT-PCR in extracts from liver (A), kidney (B) and muscle (C) of female mice from the experiment described in FIG. 21A and corresponding young (90 days old females) controls.

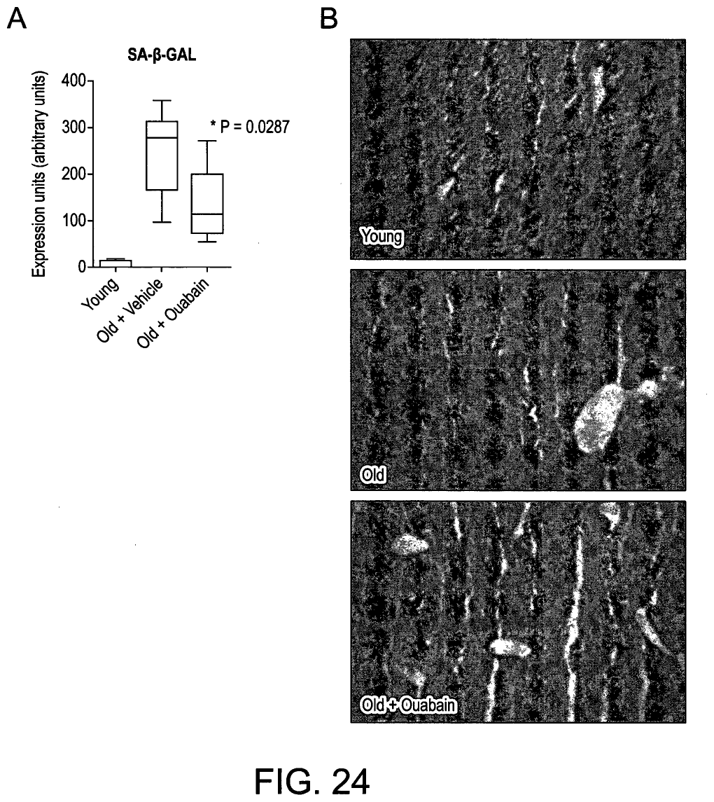

FIG. 24: Ouabain treatment of old mice results in decreased senescence-associated β-galactosidase positive cells in the liver. (A) Quantification and (B) representative pictures showing reduced SA-β-Gal activity (a marker of senesncence) in the liver of old mice treated with ouabain.

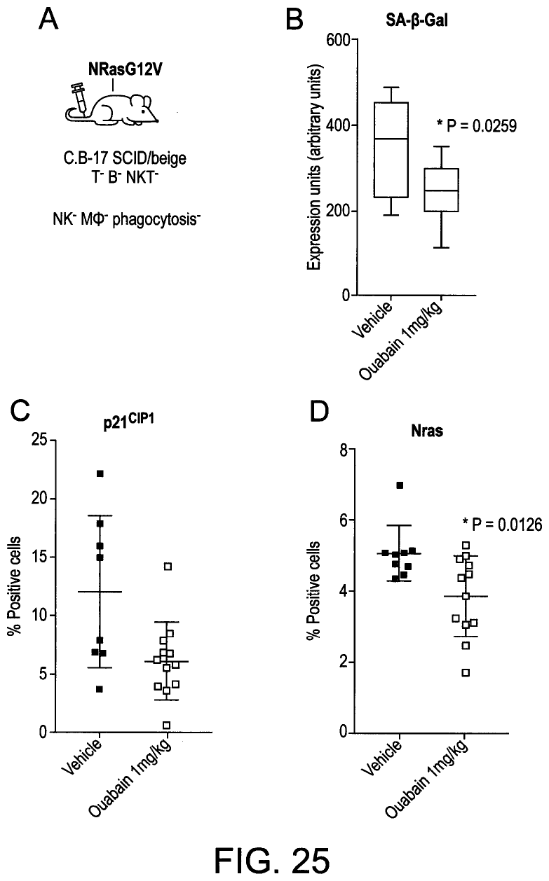

FIG. 25: Treatment with Ouabain eliminates premalignant Nras^{G12V}-positive senescent hepatocytes in a model of liver cancer initiation. (A) CB17 SCID/beige mice immunosuppressed mice were subjected to hydrodynamic tail vein injection to express NrasG12V on the liver and induce oncogene-induced senescence on hepatocytes as described in Kang et al (Nature 2011, PMID: 22080947). 5 days after mice were treated with 1 mg/kg of ouabain ip for 3 further days and livers taken the following day (day 9) for analysis. Treatment with ouabain reduced SA-b-gal activity (B), p21-positive cells (C, a marker of senescence), and Nras-positive preneoplastic hepatocytes (D).

### DETAILED DESCRIPTION

The present invention relates to agents that are capable of selectively killing senescent cells.

As used herein, the term “senolytic” refers to a compound that selectively (preferentially or to a greater degree) destroys, kills, removes, or facilitates selective destruction of senescent cells, i.e. the compound destroys or kills a senescent cell in a biologically, clinically, and/or statistically significant manner compared with its capability to destroy or kill a non-senescent cell. The senolytic compound is used in an amount and for a time sufficient to selectively kill established senescent cells, but which is insufficient to kill non-senescent cells in a clinically significant or biologically significant manner.

In certain embodiments, the senolytic compounds described herein alter at least one signaling pathway in a manner that induces (initiates, stimulates, triggers, activates, promotes) and results in death of the senescent cell. A senolytic compound may alter one or more signaling pathways in a senescent cell by interacting with one or more target proteins, which results in removing or reducing suppression of a cell death pathway, such as an apoptotic pathway. For example, contacting or exposing a senescent cell to a senolytic compound may restore the cell's mechanisms and pathways for initiating apoptosis. In one preferred embodiment, the agent induces apoptosis.

One aspect of the invention relates to an agent for use in selectively killing one or more senescent cells, wherein said agent is selected from the following: a cardiac glycoside or aglycone, a focal adhesion kinase (FAK) inhibitor, an HMG-CoA reductase inhibitor, JFD00244, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Nitazoxanide, Nitrofurazone, Temsirolimus, Eltrombopag, Adapalene, Azacyclonol, Enoxacin and Raltegravir, and pharmaceutically acceptable salts thereof.

Another aspect of the invention relates to a method of selectively killing one or more senescent cells in a subject, said method comprising administering to the subject a therapeutically effective amount of an agent selected from the following: a cardiac glycoside or aglycone, a focal adhesion kinase (FAK) inhibitor, an HMG-CoA reductase inhibitor, JFD00244, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Nitazoxanide, Nitrofurazone, Temsirolimus, Eltrombopag, Adapalene, Azacyclonol, Enoxacin and Raltegravir, and pharmaceutically acceptable salts thereof.

In a preferred aspect, the agent selectively kills senescent cells over non-senescent cells. Preferably, the agent exhibits a selectivity for killing senescent cells over non-senescent cells of at least 1.5 fold, more preferably, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 50-fold, or at least 100-fold.

In one preferred embodiment, the agent capable of selectively killing one or more senescent cells is a cardiac glycoside or aglycone.

Cardiac glycosides are a class of steroid medications used to treat heart failure. Medications containing cardiac glycosides work directly on the tissues of the heart. Cardiac glycosides consist of an aglycone (structurally related to steroid hormones) linked to one or more sugar molecules. The aglycones of cardiac glycosides can be divided into two chemical groups, the cardenolides and bufadienolides, with the former group being the most implicated in cardioactivity. The cardenolides have an unsaturated butyrolactone ring, while the bufadienolides have an α-pyrone ring. An aglycone is the compound remaining after the glycosyl group on a glycoside is replaced by a hydrogen atom. Suitable cardiac glycosides and aglycones will be familiar to a skilled person in the art.

In one preferred embodiment, the compound capable of selectively killing senescent cells is a cardiac glycoside selected from Ouabain, Digoxin and K-Stronphanthin.

In another preferred embodiment, the compound capable of selectively killing senescent cells is an aglycone selected from Strophanthidin and Bufalin.

In one highly preferred embodiment, the agent capable of selectively killing senescent cells is Ouabain. Also known as g-strophanthin, Ouabain is a cardiac glycoside and in lower doses, can be used medically to treat hypotension and some arrhythmias. It acts by inhibiting the Na/K-ATPase, also known as the sodium-potassium ion pump. Studies by the Applicant have shown that Ouabain is capable of reverting ageing associated changes in blood metabolites. Further studies have demonstrated that intermittent treatment with Ouabain improves the physical fitness of old mice (FIG. 21), and leads to reduced senscence in different organs (FIG. 22), including reduced senescence-associated 3-galactosidase activity in the liver (FIG. 24). Studies have also shown that Ouabain treatment eliminates premalignant Nras^{G12V}-positive senescent hepatocytes in a model of liver cancer initiation, thereby indicating therapeutic applications in the treatment of liver (and other) cancers.

In one preferred embodiment, the agent capable of selectively killing one or more senescent cells is an HMG-CoA reductase inhibitor.

HMG-CoA reductase is the rate-controlling enzyme of the mevalonate pathway, the metabolic pathway that produces cholesterol and other isoprenoids. Normally in mammalian cells this enzyme is suppressed by cholesterol derived from the internalization and degradation of low density lipoprotein via the LDL receptor as well as oxidized species of cholesterol. Competitive inhibitors of the reductase induce the expression of LDL receptors in the liver, which in turn increases the catabolism of plasma LDL and lowers the plasma concentration of cholesterol, an important determinant of atherosclerosis. This enzyme is therefore the target of the widely available cholesterol-lowering drugs known collectively as the statins.

HMG-CoA reductase inhibitors can be identified by known laboratory assays (Gerber et al., 2004), or using commercially available assay kits (e.g. Sigma Aldrich). Preferably, for the purposes of the present invention, a compound is considered to be an inhibitor of HMG-CoA reductase if it exhibits an IC₅₀ value in the above assay of less than about 100 μM, more preferably, less than about 50 μM, even more preferably, less than about 25 μM, less than about 10 μM, less than about 5 μM, less than about 1 μM, less than about 0.1 μM, or less than about 0.01 μM, or less than about 1 nM.

In one preferred embodiment, the HMG-CoA reductase inhibitor is a statin. Examples of statins include rosuvastatin (CRESTOR), lovastatin (Mevacor), atorvastatin (Lipitor), pravastatin (Pravachol), fluvastatin (Lescol), pitavastatin (Livalo), and simvastatin (Zocor). Red yeast rice extract, one of the fungal sources from which the statins were discovered, contains several naturally occurring cholesterol-lowering molecules known as monacolins. The most active of these is monacolin K, or lovastatin (previously sold under the trade name Mevacor, and now available as generic lovastatin). HMG-CoA reductase inhibitors (e.g. statins), are used to lower serum cholesterol as a means of reducing the risk for cardiovascular disease.

In a more preferred embodiment, the statin is selected from Atorvastatin calcium, Fluvastatin sodium, Lovastatin, Pitavastatin calcium and Simvastatin.

Statins such as Simvastatin have been reported to decrease the senescence-associated secretory phenotype (SASP) of senescent human fibroblasts by inhibiting protein prenylation without affecting senescent growth arrest or eliminating the firbroblast via apoptosis (Lui et al, Sci Rep 2015 Dec. 14; 5:17895. doi: 10.1038/srep17895). Further studies report that HMG-CoA reductase inhibitors induce apoptosis in dividing cells, such as lymphoma cells, by promoting ROS generation and regulating Akt, Erk and p38 signals via suppression of the mevalonate pathway (Qi et al, Cell Death and Disease (2013) 4, e518; doi:10.1038/cddis.2013.44). Surprisingly, studies by the present applicant have now shown that statins have the ability to kill senescent cells.

In another embodiment of the invention, the agent capable of selectively killing one or more senescent cells is a focal adhesion kinase (FAK) inhibitor.

Focal adhesion kinase (FAK), also known as protein tyrosine kinase 2 (PTK2), is a protein that in humans is encoded by the PTK2 gene. PTK2 is a focal adhesion-associated protein kinase involved in cellular adhesion and spreading processes.

FAK is a protein of 125 kD recruited as a participant in focal adhesion dynamics between cells, and has a role in motility and cell survival. FAK is a highly conserved, non-receptor tyrosine kinase originally identified as a substrate for the oncogene protein tyrosine kinase v-src. This cytosolic kinase has been implicated in diverse cellular roles including cell locomotion, mitogen response and cell survival. FAK is typically located at structures known as focal adhesions, which are multi-protein structures that link the extracellular matrix (ECM) to the cytoplasmic cytoskeleton. Additional components of focal adhesions include actin, filamin, vinculin, talin, paxillin, tensin and RSU-1.

FAK inhibitors can be identified by known laboratory assays, using commercially available kits, for example ADP-GIo™ kinase assay (Promega) or FAK (Phospho)[pT937]] Human ELISA kit (Thermo Fisher Scientific). Preferably, for the purposes of the present invention, a compound is considered to be a FAK inhibitor if it exhibits an IC₅₀ value in the above assay of less than about 100 μM, more preferably, less than about 50 μM, even more preferably, less than about 25 μM, less than about 10 μM, less than about 5 μM, less than about 1 μM, less than about 0.1 μM, or less than about 0.01 μM, or less than about 1 nM.

In a preferred embodiment, the focal adhesion kinase (FAK) inhibitor is selected from PF-573228, NSC 677249 and Defactinib.

In another embodiment, the agent capable of selectively killing one or more senescent cells is selected from the following:

| Name | Structure |
|---|---|
| PF-573228 6-[4-(3-methyane- sulfonyl-benzyl- amino)-5-trifluoro- methyl-pyrimidin-2- ylamino]-3,4-dihydro- 1H-quinolin-2-one | 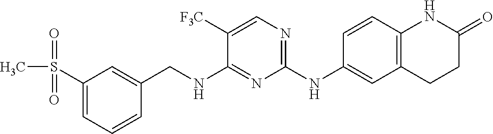 [SMILES: CS(=O)(=O)c1cccc(CNc2nc(Nc3ccc4c(c3)CCC(=O)N4)ncc2C(F)(F)F)c1] |
| JFD00244 1,4-Bis[(p-hydroxy- phenethyl)amino]- anthraquinone | 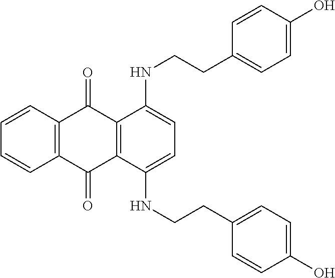 [SMILES: O=C1c2ccccc2C(=O)c2c(NCCc3ccc(O)cc3)ccc(NCCc3ccc(O)cc3)c21] |
| Ouabain (Strodival) | 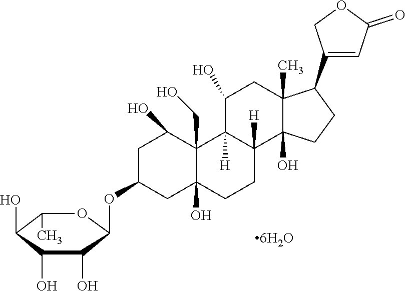 |
| Cyclosporine (Neoral) | 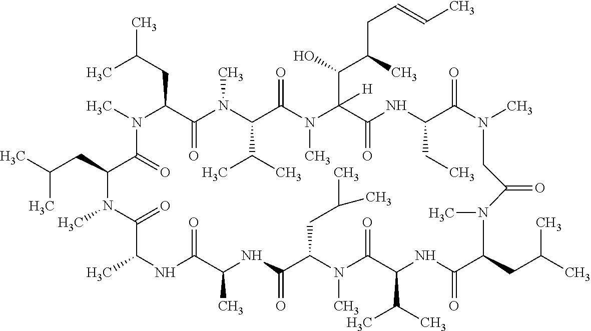 |
| Tyrphostin AG879 (2E)-3-[3,5-Bis(1,1- dimethylethyl)-4- hydroxyphenyl]-2- cyano-2- propenethioamide | 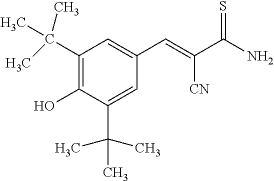 [SMILES: CC(C)(C)c1cc(/C=C(\C#N)C(N)=S)cc(C(C)(C)C)c1O] |
| Cantharidin 2,6-dimethyl-4,10- dioxatricyclo- [5.2.1.0^{2,6}]decane-3,5- dione or alpha-cyano- (3,5-di-t-butyl-4- hydroxy)thiocinnamide | 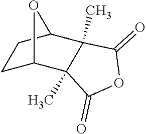 |
| Diphenyleneiodonium chloride (DPI) | 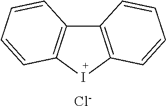 |
| Rottlerin (Mallotoxin) | 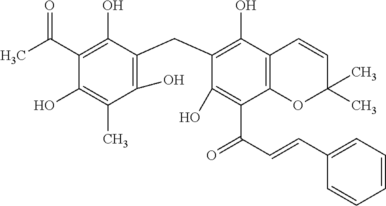 [SMILES: CC(=O)c1c(O)c(C)c(O)c(Cc2c(O)c3c(c(C(=O)/C=C/c4ccccc4)c2O)OC(C)(C)C=C3)c1O] |
| 2,3-Dimethoxy-1,4- naphthoquinone (DMNQ) | 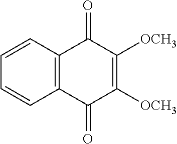 [SMILES: COC1=C(OC)C(=O)c2ccccc2C1=O] |
| LY-367,265 | 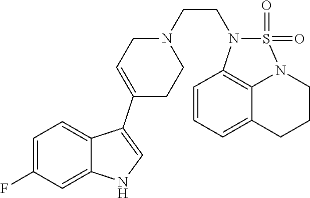 [SMILES: O=S1(=O)N(CCN2CC=C(c3c[nH]c4cc(F)ccc34)CC2)c2cccc3c2N1CCC3] |
| Rotenone (Dactinol, Paraderil; Tubatoxin) (2R,6aS,12aS)-1,2,6, 6a,12,12a-hexahydro-2- isopropenyl-8,9- dimethoxychromeno[3,4- b]furo(2,3-h)chromen-6- one | 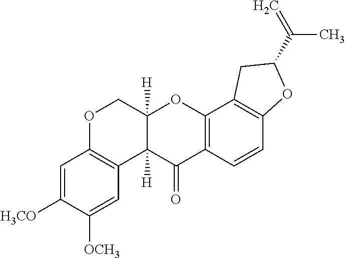 [SMILES: C=C(C)[C@H]1Cc2c(ccc3c2O[C@@H]2COc4cc(OC)c(OC)cc4[C@@H]2C3=O)O1] |
| Idarubicin (Zavedos, Idamycin) | 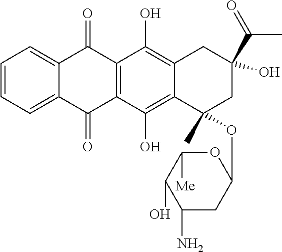 |
| Dequalinium Chloride | 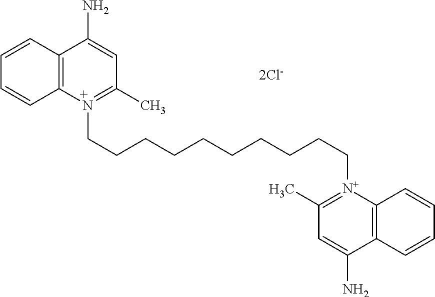 |
| Vincristine (Oncovin, Vincasar) | 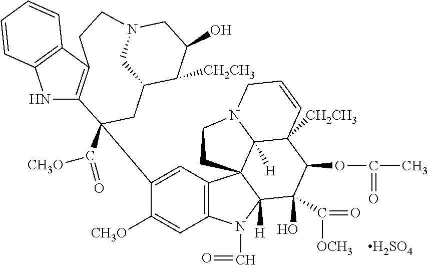 |
| Atorvastatin calcium (Lipitor) | 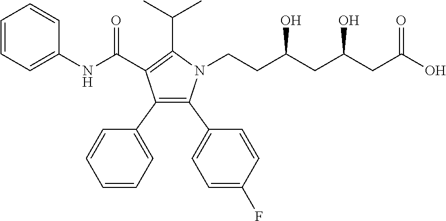 [SMILES: CC(C)c1c(C(=O)Nc2ccccc2)c(-c2ccccc2)c(-c2ccc(F)cc2)n1CC[C@@H](O)C[C@@H](O)CC(=O)O] |
| Fluvastatin Sodium (Lescol) | 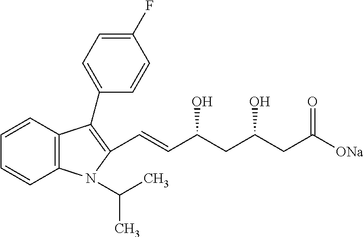 |
| Lovastatin (Mevacor) | 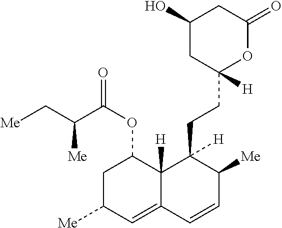 |
| Pitavastatin Calcium (Livalo, Livazo) | 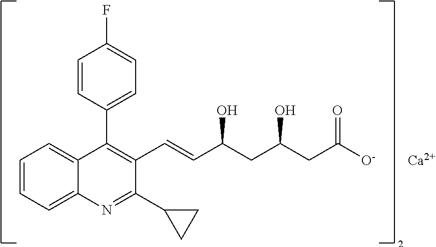 |
| Simvastatin (Zocor) | 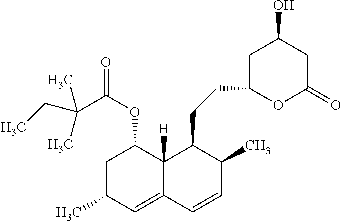 [SMILES: CCC(C)(C)C(=O)O[C@H]1C[C@@H](C)C=C2C=C[C@H](C)[C@H](CC[C@@H]3C[C@@H](O)CC(=O)O3)[C@H]21] |
| Nitazoxanide (Alinia, Annita) | 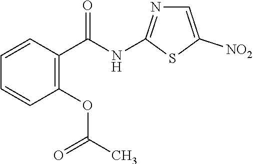 [SMILES: CC(=O)Oc1ccccc1C(=O)Nc1ncc([N+](=O)[O-])s1] |
| Nitrofurazone (Nitrofural, Furacin) | 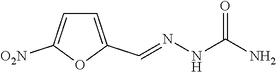 [SMILES: NC(=O)N/N=C/c1ccc([N+](=O)[O-])o1] |
| Temsirolimus (Torisel) | 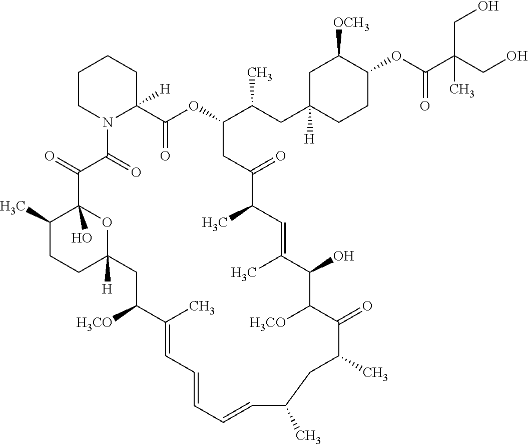 [SMILES: COC1C(=O)[C@H](C)C[C@H](C)/C=C/C=C/C=C(\C)[C@@H](OC)C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)C(C)(CO)CO)[C@H](OC)C2)CC(=O)[C@H](C)/C=C(\C)[C@H]1O] |
| Eltrombopag (SB-497115-GR) (Promacta, Revolade) | 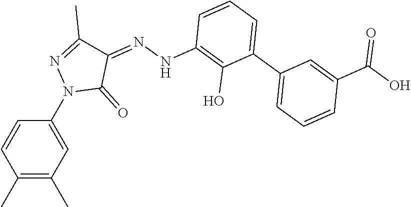 [SMILES: CC1=NN(c2ccc(C)c(C)c2)C(=O)/C1=N\Nc1cccc(-c2cccc(C(=O)O)c2)c1O] |
| Adapalene (Differin) | 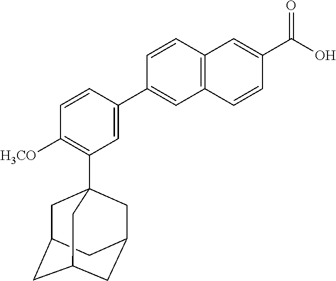 [SMILES: COc1ccc(-c2ccc3cc(C(=O)O)ccc3c2)cc1C12CC3CC(CC(C3)C1)C2] |
| Azacyclonol (Ataractan) | 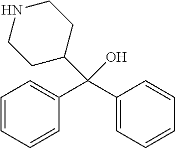 [SMILES: OC(c1ccccc1)(c1ccccc1)C1CCNCC1] |
| Enoxacin (Penetrex) | 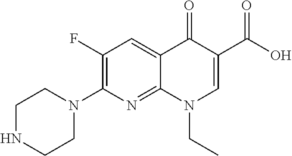 [SMILES: CCn1cc(C(=O)O)c(=O)c2cc(F)c(N3CCNCC3)nc21] |
| Raltegravir (MK-0518) (Isentress) | 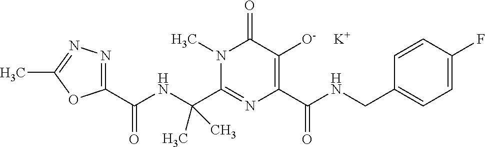 [SMILES: Cc1nnc(C(=O)NC(C)(C)c2nc(C(=O)NCc3ccc(F)cc3)c([O-])c(=O)n2C)o1 ; [K+]] |
| NSC 677249 (1,2,4,5- Benzenetetramine tetrahydrochloride) | 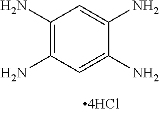 |
| Defactinib (VS-6063, PF- 04554878) | 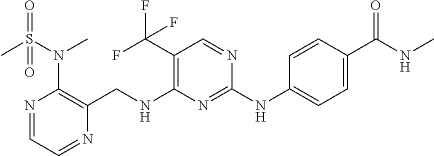 |
| Digoxin (Lanoxin) | 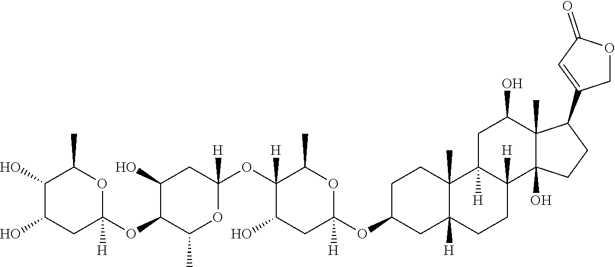 |
| Bufalin (3β,5β)-3,14- Dihydroxybufa-20,22- dienolide | 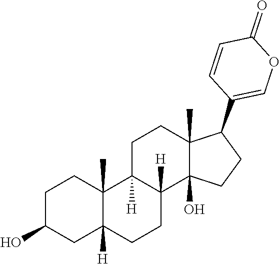 |
| K-strophanthin (Cymarin) | 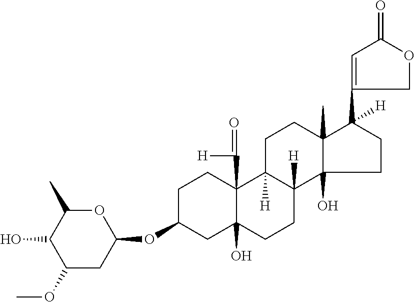 |
| Strophanthidin (Cymarigenen) | 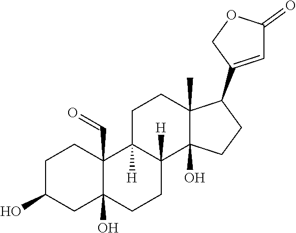 |

Another aspect of the invention relates to a senolytic agent as defined in the above table.

Another aspect of the invention relates to an agent as defined herein for use as a senolytic. Preferably, the agent is as defined in the above table.

The senolytic activity of the compounds according to the invention is demonstrated with reference to the accompanying examples, and is described in more detail below.

Studies by the Applicant in IMR90 ER:RAS cells have demonstrated that the compounds described herein can eliminate senescent cells. In particular, the compounds resulted in a selective reduction of senescent (but not normal) cell numbers and are therefore candidate senolytic agents.

In the setup used, IMR90 ER:RAS cells were treated with 100 nM 4OHT to induce OIS (oncogene induced senescence) and activate the ER:RAS fusion protein. 6 days after 4OHT induction (+4OHT), once cells were fully senescent, the agents to be tested were added at a concentration of 10 μM, and cell numbers were assessed 4 days after by DAPI stained couple to automated microscopy. Alternatively, cells were fixed and stained with crystal violet. In parallel, the same treatments were carried out in IMR90 ER:RAS cells treated with DMSO (−4OHT). These cells do not undergo senescence. A plot of cell numbers (−4OHT) versus (+4OHT) identified agents capable of selectively eliminating senescent cells. Amongst the candidates tested, tyr-AG879, PF-573228 and Cyclosporine showed the most promising senolytic properties (FIG. 1). The selective killing of senescent cells in in IMR90 ER:RAS by further compounds according to the invention is confirmed in FIG. 10.

To validate their role as senolytics, the compounds were tested for their ability to selectively eliminate different types of senescent cells when used at 3 different concentrations (1, 5 and 10 μM). A summary of the different cell culture models of senescence used to test these compounds is presented in FIG. 2. In these experiments the compounds were tested side by side with a control ABT-263 (the de facto gold standard senolytic compound) (Chang et al., 2016; Yosef et al., 2016; Zhu et al., 2016). Experiments were conducted to test the ability of the compounds to behave as senolytics during OIS (using IMR90 ER:RAS, FIG. 3 and HMEC hTERT ER:RAS, FIG. 4). The results suggest that the senolytic compounds not only behave as senolytic in human fibroblasts, but also in other cell types. To understand whether the selective killing of senescent IMR90 ER:RAS was a consequence of senescence induction or of activation of RAS signaling, experiments were conducted in isogenic IMR90 ER:RAS E6/E7 cells. In these cells, RAS signalling is induced by adding 100 nM 4OHT that activates the ER:RAS fusion protein. However, expression of the E6/E7 protein prevents senescence induction. Two of the tested compounds, Ouabain and Diphenyleneiodonium, displayed selectivity for killing IMR90 ER:RAS E6/E7 (FIG. 5). Although this suggests that part of the selectivity of these compound is not due to senescence activation but to RAS signalling, since hyperactivation of RAS is a hallmark of many tumours these compounds are still interesting.

Experiments were conducted to determine whether compounds according to the invention could also behave as senolytic agents when senescence is induced by stimuli other than oncogene activation. Compounds were tested for their ability to eliminate cells undergoing replicative senescence (old IMR90 cells, FIG. 6), and senescence induced by chemotherapeutic drugs such as etoposide (FIG. 7) and doxorubicin (FIG. 8). A summary of the results is presented in FIG. 9. While some compounds demonstrated a broad spectrum (behaving as senolytics in many of the systems used), others were more specific to a given cell type or inducer.

One compound, PF-573228, was a FAK inhibitor. Experiments were conducted to test whether other FAK inhibitors also behaved as senolytics. Compound 14 (also known as NSC 677249) and Defactinib were also shown to have senolytic properties (FIG. 12). These results suggest that FAK is a gene essential for senescent cells and that FAK inhibitors could therefore have therapeutic applications as senolytic agents. Various statins were also identified as being senolytic. Statins are HMG-CoA reductase inhibitors and are a class of lipid-lowering medications used to prevent cardiovascular disease (Goldstein and Brown, 2015). The senolytic effect of 5 of these statins was confirmed experimentally (FIG. 11A). Further experiments served to confirm the dose dependent effect of atorvastatin and simvastatin as senolytics (FIG. 11B-C). These experiments suggest that HMG-CoA reductase is an essential gene for senescent cells, thus inhibitors of HMG-CoA reductase have therapeutic applications as senolytic agents.

Experiments were conducted to investigate the mechanism by which the senolytics caused the elimination of senescent cells using a pan-caspase inhibitor (QVD). Addition of this inhibitor prevented the elimination of senescent cells by PF-573228, rottlerin and rotenone, indicating that these drugs selectively induce apoptosis of senescent cells (FIG. 13).

The above experiments were all performed comparing the effects of the drugs in cultures of normal and senescent cells. To understand whether the drugs selectively kill senescent cells from a mixed culture, experiments were conducted using Cherry-labelled normal IMR90 cells and GFP labelled IMR90 ER:RAS cells that could be induced to undergo senescence upon treatment with 4OHT (FIG. 14A). Using this system, senolytics JFD 00244 and Rottlerin result in the selective elimination of senescent cells from the co-cultures in a similar manner to ABT-263 (FIG. 14B-C).

Further experiments investigated whether the senolytic agents were capable of eliminating senescent cells in vivo. Treatment of mice with the chemotherapeutic agent Doxorubicin has been shown to induce senescence. These senescent cells are responsible for some of the side-effects associated with chemotherapy and can fuel the growth of cancerous cells (Demaria et al., 2016). To understand if treatment with Ouabain could eliminate senescent cells induced by doxorubicin, experiments were carried out as described in FIG. 15. Doxorubicin treatment induces senescence in the lung. Treatment with Ouabain resulted in the partial elimination of senescent cells induced by Doxorubicin treatment of mice (FIG. 15).

Whole-body irradiation is known to cause senescence. In irradiated mice, senescent cells accumulate in different tissues, including the lung (Chang et al., 2016; Yosef et al., 2016). This can be partially prevented upon treatment with the known senolytic ABT-263 (Chang et al., 2016; Yosef et al., 2016). Treatment with either Ouabain or DPI partially eliminated these senescent cells in a similar manner to ABT-263 (FIG. 16).

In summary, experiments by the Applicant have identified a range of senolytic agents. Their effects have been validated across different systems of senescence and have identified that while some of these senolytics have a broad specificity, others are more selective for cell type or senescent inducer. Initial experiments identified that multiple inhibitors against two targets (FAK and HMG-CoA reductase) behave as senolytics. The data suggest that FAK and HMG-CoA reductase are genes essential for senescent cells. Thus, agents which inhibit FAK or HMG-CoA reductase have therapeutic applications as senolytic agents. Experiments have also confirmed that treatment with Ouabain and DPI partially eliminates senescent cells in mice, suggesting that these agents (and other senolytics identified herein) have applications in the treatment of senescence-related diseases.

### Therapeutic Applications

The present invention relates to agents for use in selectively killing one or more senescent cells. Further aspects of the invention relate to compounds for use in treating or preventing senscence-associated diseases or disorders, and methods relating thereto.

In one preferred embodiment, the agent selectively kills cells undergoing oncogene-induced senescence.

In another preferred embodiment, the agent selectively kills cells undergoing drug-induced senescence.

In another preferred embodiment, the agent selectively kills cells undergoing irradiation-induced senescence.

Depending on the nature of the pharmaceutically active agent, the agents described herein can be used to treat a wide range of different senescence-associated diseaess and disorders.

For example, in one preferred embodiment, the agent delays tumorigenesis. In another embodiment, the agent attenuates cataract formation. In another embodiment, the agent attenuates atherosclerosis. In another embodiment, the agent attenuates the age-related deterioration of kidney, fat and heart amongst other organs.

One aspect of the invention relates to a compound for use in treating or preventing a senescence-associated disease or disorder, wherein said compound is a cardiac glycoside or aglycone, and said senescence-associated disease or disorder is other than a cardiovascular disorder. Preferably, for this aspect, the cardiac glycoside or aglycone is selected from Ouabain, Bufelin, Digoxin, K-Strophanthin and Strophanthidin.

Another aspect of the invention relates to a compound for use in treating or preventing a senescence-associated disease or disorder, wherein said compound is an HMG-CoA reductase inhibitor, and said senescence-associated disease or disorder is other than a cardiovascular disorder. Preferably, the disorder is a proliferative disorder. Preferably, for this aspect, the HMG-CoA reductase inhibitor is a statin, more preferably, a statin selected from Atorvastatin calcium, Fluvastatin sodium, Lovastatin, Pitavastatin calcium and Simvastatin.

Another aspect of the invention relates to a compound for use in treating or preventing a senescence-associated disease or disorder, wherein said compound is a focal adhesion kinase (FAK) inhibitor. Preferably, for this aspect, the focal adhesion kinase (FAK) inhibitor is selected from PF-573228, NSC 677249 and Defactinib. Preferably, for this embodiment, the disorder is other than a proliferative disorder (e.g. cancer).

Another aspect of the invention relates to a compound for use in treating or preventing a senescence-associated disease or disorder, wherein said compound is selected from the following: JFD00244, Cyclosporine, Tyrphostin AG879, Cantharidin, Diphenyleneiodonium chloride, 2,3-Dimethoxy-1,4-naphthoquinone, LY-367,265, Rotenone, Idarubicin, Dequalinium chloride, Vincristine, Atorvastatin calcium, Nitazoxanide, Nitrofurazone, Temsirolimus, Eltrombopag, Adapalene, Azacyclonol, Enoxacin and Raltegravir, and pharmaceutically acceptable salts thereof (where the compound is not already in salt form).

Another aspect of the invention relates to a compound for use in treating or preventing a senescence-associated disease or disorder, wherein said senescence-associated disease or disorder is other than a cardiovascular disorder, and wherein said compound is selected from the following: PF-573228, JFD00244, Ouabain, Bufalin, Digoxin, K-Strophanthin, Strophanthidin, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin, and Raltegravir, and pharmaceutically acceptable salts thereof (where the compound is not already in salt form).

Another aspect of the invention relates to a a method for treating or preventing a senescence-associated disease or disorder other than a cardiovascular disorder, said method comprising administering to a subject a therapeutically effective amount of an HMG-CoA reductase inhibitor.

Another aspect of the invention relates to a method for treating or preventing a senescence-associated disease or disorder, said method comprising administering to a subject a therapeutically effective amount of a focal adhesion kinase (FAK) inhibitor. Preferably, for this embodiment, the disorder is other than a proliferative disorder (e.g. cancer).

Another aspect of the invention relates to a method for treating or preventing a senescence-associated disease or disorder by selectively killing senescent cells, said method comprising administering to a subject a therapeutically effective amount of a compound selected from the following: PF-573228, JFD00244, Ouabain, Bufalin, Digoxin, K-Strophanthin, Strophanthidin, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin, Raltegravir, NSC 677249 and Defactinib, and pharmaceutically acceptable salts thereof (where the compound is not already in salt form).

Preferably, the compound is selected from the following: PF-573228, JFD00244, Ouabain, Bufalin, Digoxin, K-Strophanthin, Strophanthidin, Cyclosporine, Tyrphostin AG879, Cantharidin, Diphenyleneiodonium chloride, Rottlerin, 2,3-Dimethoxy-1,4-naphthoquinone, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Eltrombopag, Adapalene, NSC 677249 and Defactinib, and pharmaceutically acceptable salts thereof (where the compound is not already in salt form).

Another aspect of the invention relates to the use of a compound selected from the following: PF-573228, JFD00244, Ouabain, Bufalin, Digoxin, K-Strophanthin, Strophanthidin, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin, Raltegravir, NSC 677249 and Defactinib, and pharmaceutically acceptable salts thereof, where the compound is not already in salt form;

in the preparation of a medicament for treating or preventing a senescence-associated disease or disorder, wherein said compound selectively kills one or more senescent cells.

Another aspect of the invention relates to the use of cardiac glycoside or aglycone in the preparation of a medicament for treating or preventing a senescence-associated disease or disorder other than a cardiovascular disorder. Preferably, the disorder is a proliferative disorder as detailed below.

Another aspect of the invention relates to the use of a focal adhesion kinase (FAK) inhibitor in the preparation of a medicament for treating or preventing a senescence-associated disease or disorder. Preferably, the disorder is other than a proliferative disorder (e.g. cancer).

Another aspect of the invention relates to the use of an HMG-CoA reductase inhibitor in the preparation of a medicament for treating or preventing a senescence-associated disease or disorder, wherein said senescence-associated disease or disorder is other than a cardiovascular disorder.

Senescent cells can be detected by techniques and procedures known in the art. For example, the presence of senescent cells can be analyzed by histochemical or immunohistochemical techniques that detect the senescence marker, SA-beta galactosidase (SA-β-gal) (Dimri et al, Proc. Natl. Acad. Sci. USA 92: 9363-9367 (1995); see also methods described in WO 2015/116735). Alternatively, senescent cells can be detected by the presence of the senescent cell-associated polypeptide p16, as determined by known immunochemical methods, for example, immunoblotting analysis. Expression of pI6 mRNA in a cell can be measured by a variety of techniques, including quantitative PCR. Senescence cell associated polypeptides can be detected using known assays, for example, a Luminex array assay (Coppe et al, PLoS Biol 6: 2853-68 (2008)).

The presence of senescent cells can also be determined by detecting other senescent cell-associated molecules, such as cytokines, chemokines, expressed and secreted growth factors, proteases, cell-related metabolites, reactive oxygen species, and other molecules that stimulate inflammation and/or other biological effects. Examples of senescent cell-associated molecules include those that comprise the senescence-associated secretory phenotype (SASP), senescent-messaging secretome, and DNA damage secretory program (DDSP).

As used herein, senescence-associated disorders or diseases include disorders or diseases associated with, or caused by cellular senescence, including age-related diseases and disorders. A senescence-associated disease or disorder may also be called a senescent cell-associated disease or disorder. A prominent feature of aging is a gradual loss of function, or degeneration that occurs at the molecular, cellular, tissue, and organismal levels. Age-related degeneration gives rise to well-recognized pathologies such as sarcopenia, atherosclerosis and heart failure, osteoporosis, pulmonary insufficiency, renal failure, neurodegeneration (including macular degeneration, Alzheimer's disease, and Parkinson's disease), and many others.

Senescence-associated diseases and disorders include, but are not limited to, cardiovascular diseases and disorders, proliferative disorders such as cancer, inflammatory diseases and disorders, autoimmune diseases and disorders, pulmonary diseases and disorders, eye diseases and disorders, metabolic diseases and disorders, neurological diseases and disorders (e.g., neurodegenerative diseases and disorders); age-related diseases and disorders induced by senescence; skin conditions; age-related diseases; dermatological diseases and disorders; and transplant related diseases and disorders.

Preferably, the subject is a mammal, more preferably a human.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein, the term “treating” includes abrogating, substantially inhibiting, slowing or reversing the progression of a disease or disorder, substantially ameliorating clinical symptoms of a disease or disorder or substantially preventing the appearance of clinical symptoms of a disease or disorder.

Herein, the term “preventing” refers to a method for barring an organism from acquiring a disorder or disease in the first place.

The term “therapeutically effective amount” refers to that amount of the agent being administered which will relieve to some extent one or more of the symptoms of the disease or disorder being treated.

For any agent used in this invention, a therapeutically effective amount, also referred to herein as a therapeutically effective dose, can be estimated initially from cell culture assays. For example, a dose can be formulated in animal models to achieve a circulating concentration range that includes the IC₅₀ or the IC₁₀₀ as determined in cell culture. Such information can be used to more accurately determine useful doses in humans. Initial dosages can also be estimated from in vivo data. Using these initial guidelines one of ordinary skill in the art could determine an effective dosage in humans.

Moreover, toxicity and therapeutic efficacy of the agents described herein can be determined by standard pharmaceutical procedures in cell cultures or experimental animals, e.g., by determining the LD₅₀ and the ED₅₀. The dose ratio between toxic and therapeutic effect is the therapeutic index and can be expressed as the ratio between LD₅₀ and ED₅₀. Compounds which exhibit high therapeutic indices are preferred. The data obtained from these cell cultures assays and animal studies can be used in formulating a dosage range that is not toxic for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (see, e.g., Fingl et al, 1975, In: The Pharmacological Basis of Therapeutics, chapter 1, page 1).

Dosage amount and interval may be adjusted individually to provide plasma levels of the active compound which are sufficient to maintain therapeutic effect. Preferably, therapeutically effective serum levels will be achieved by administering multiple doses each day. In cases of local administration or selective uptake, the effective local concentration of the drug may not be related to plasma concentration. One skilled in the art will be able to optimize therapeutically effective local dosages without undue experimentation.

One aspect of the invention relates to a cardiac glycoside or aglycone for use in preventing or treating a senescence-associated disorder or disease, wherein said cardiac glycoside or aglycone selectively kills one or more senescent cells. Preferably, for this aspect, the senescence-associated disorder or disease is not a cardiovascular disorder.

One aspect of the invention relates to an HMG-CoA reductase inhibitor for use in preventing or treating a senescence-associated disorder or disease, wherein said HMG-CoA reductase inhibitor selectively kills one or more senescent cells. Preferably, for this aspect, the senescence-associated disorder or disease is not a cardiovascular disorder.

Another aspect of the invention relates to a FAK inhibitor for use in preventing or treating a senescence-associated disorder or disease, wherein said FAK inhibitor selectively kills one or more senescent cells. Preferably, for this aspect, the senescence-associated disorder or disease is not a proliferative disorder such as cancer. For this aspect, preferably the senescence-associated disease or disorder is a cardiovascular disease or disorder, a metabolic disease, an inflammatory disease or disorder, a pulmonary disease or disorder, or a neurological disease or disorder.

### Proliferative Disorders

In one preferred embodiment of the invention, the agent can be used in the treatment of a proliferative disorder such as cancer or leukemia.

Non-limiting examples of cancers which may be treated according to this aspect of the invention include: adenocarcinoma, adrenal gland tumor, ameloblastoma, anaplastic, anaplastic carcinoma of the thyroid, angiofibroma, angioma, angiosarcoma, apudoma, argentaffmoma, arrhenoblastoma, ascites tumor cell, ascitic tumor, astroblastoma, astrocytoma, ataxia-telangiectasia, atrial myxoma, a basal cell carcinoma cell, bone cancer, brainstem glioma, brain tumor, breast cancer, Burkitt's lymphoma, cerebellar astrocytoma, cervical cancer, cherry angioma, cholangiocarcinoma, cholangioma, chondroblastoma, chondroma, chondrosarcoma, chorioblastoma, choriocarcinoma, colon cancer, common acute lymphoblastic leukemia, craniopharyngioma, cystocarcinoma, cystofbroma, cystoma, ductal carcinoma in situ, ductal papilloma, dysgerminoma, encephaloma, endometrial carcinoma, endothelioma, ependymoma, epithelioma, erythroleukemia, Ewing's sarcoma, extra nodal lymphoma, feline sarcoma, fibro adenoma, fibro sarcoma, follicular cancer of the thyroid, ganglioglioma, gastrinoma cell, glioblastoma multiform, glioma, gonadoblastoma, haemangioblastoma, haemangioendothelioblastoma, haemangioendothelioma, haemangiopericytoma, haematolymphangioma, haemocytoblastoma, haemocytoma, hairy cell leukemia, hamartoma, hepatocarcinoma, hepatocellular carcinoma, hepatoma, histoma, Hodgkin's disease, hypernephroma, infiltrating cancer, infiltrating ductal cell carcinoma, insulinoma, juvenile angioforoma, Kaposi sarcoma, kidney tumor, large cell lymphoma, leukemia, a leukemia, acute leukemia, lipoma, liver cancer, liver metastases, Lucke carcinoma, lymphadenoma, lymphangioma, lymphocytic leukemia, lymphocytic lymphoma, lymphoeytoma, lymphoedema, lymphoma, lung cancer, malignant mesothelioma, malignant teratoma, mastocytoma, medulloblastome, melanoma, meningioma, mesothelioma, Morton's neuroma, multiple myeloma, myeloblastoma, myeloid leukemia, myelolipoma, myeloma, myoblastoma, myxoma, nasopharyngeal carcinoma, neoplastic, nephroblastoma, neuroblastoma, neurofibroma, neurofibromatosis, neuroglioma, neuroma, non-Hodgkin's lymphoma, oligodendroglioma, optic glioma, osteochondroma, osteogenic sarcoma, osteosarcoma, ovarian cancer, Paget's disease of the nipple, pancoast tumor, pancreatic cancer, phaeochromocytoma, pheoehromocytoma, plasmacytoma, primary brain tumor, progonoma, prolactinoma, renal cell carcinoma, retinoblastoma, rhabdomyosarcoma, rhabdo sarcoma, a solid tumor, sarcoma, a secondary tumor, seminoma, skin cancer, small cell carcinoma, squamous cell carcinoma, strawberry haemangioma, T-cell lymphoma, teratoma, testicular cancer, thymoma, trophoblastic tumor, Wilm's tumor.

In one highly preferred embodiment, the agent of the invention can be used in conjunction with other treatments for cancer that induce senescence, such as irradiation or chemotherapy (for example, treatment with Palbociclib, ribociclib or abemaciclib, or other chemotherapeutic agents). Thus, by treating at the same time, or after the other treatment, the agent can:
eliminate cancer cells that have been pushed to senescence; and/or
eliminate or reduce certain side effects produced by senescent cells such as inflammation, promotion of cancer growth, promotion of metastasis and other side effects of chemotherapy or radiotherapy; and/or
reduce or eliminate precancerous lesions; and/or
eliminate or reduce cells that undergo senescence by treatment with CDK4/6 inhibitors.

In one preferred embodiment, the agent can reduce or eliminate precancerous (or pre-neoplastic) lesions. Senescent cells exist in premalignant tumours, but not in malignant ones. In this regard, it is understood that a substantial number of cells in premalignant tumours undergo oncogene-induced senescence, but that cells in malignant tumours are unable to do this owing to the loss of oncogene-induced senescence effectors such as p16^{INK4a} Or p53 (Collado et al; Nature, Vol 436, August 2005, page 642). Thus, one embodiment of the invention relates to an agent as described herein for use in reducing or eliminating precancerous (or pre-neoplastic) lesions.

In another preferred embodiment of the invention, the agent can eliminate or reduce chemotherapy-induced senescence, for example, before, during or after treatment with a chemotherapeutic agent. Chemotherapeutic agents include, but are not limited to, anthracyclines, doxorubicin, etoposide, daunorubicin, taxols, paclitaxel, gemcitabine, pomalidomide, and lenalidomide. Thus, in one preferred embodiment, the agent can be used in combination treatment with a chemotherapeutic agent, where the agent is administered separately, sequentially or concomitantly with the chemotherapeutic agent. One embodiment of the invention relates to an agent as described herein for use in eliminating or reducing chemotherapy-induced senescence.

In another preferred embodiment of the invention, the agent can eliminate or reduce senescence induced by treatment with a CDK inhibitor, for example, a CDK4 or CDK6 inhibitor. Thus, in one preferred embodiment, the agent can be used in combination treatment with a CDK4 or CDK6 inhibitor, where the agent is administered separately, sequentially or concomitantly with the CDK4 or CDK6 inhibitor.

In another preferred embodiment of the invention, the agent can eliminate or reduce Palbociclib-induced senescence. In the context of eliminating or reducing Palbociclib-induced senescence, administration of the agent can potentially prevent cancer remission as cells reenter the cell cycle (see Cadoo et al; Breast Cancer: Targets and Therapy 2014; 6, 123-133).

In another preferred embodiment, the agent can eliminate cancer cells that have been pushed to senescence. In one preferred embodiment, the agent delays tumorigenesis.

In another preferred embodiment, the agent can eliminate or reduce certain side effects produced by senescent cells such as inflammation, promotion of cancer growth, promotion of metastasis and other side effects of chemotherapy or radiotherapy. Although cellular senescence suppresses tumorigenesis early in life, studies have shown that it may promote cancer in aged organisms (Krtolica et al; PNAS, Oct. 9, 2001, Vol 98, No 21, 12072-12077). Oncogene-induced senescence is classically considered a tumour defense barrier. However, several studies have shown that under certain circumstances, senescent cells may favour tumour progression because of their secretory phenotype (Angelini et al; Cancer Res 73(1), 1 Jan. 2013, 450-458).

In another preferred embodiment, the agent can decrease inflammation that drives cancer and metastasis.

In one preferred embodiment, the agent can be used in the treatment of a chemotherapy-induced or radiotherapy-induced side effect. Non-limiting examples of chemotherapeutic agents include anthracyclines, doxorubicin, etoposide, daunorubicin, taxols, paclitaxel, gemcitabine, pomalidomide, and lenalidomide. Chemotherapy-induced side effects or radiotherapy-induced side effects include, but art not limited to, weight loss, endocrine changes, hormone imbalance, changes in hormone signaling, changes is cardiotoxicity, body composition, reduced ability to be physically active, gastrointestinal toxicity, nausea, vomiting, constipation, anorexia, diarrhea, peripheral neuropathy, fatigue, malaise, low physical activity, hematological toxicity, anemia, hepatotoxicity, alopecia, pain, infection, mucositis, fluid retention, dermatological toxicity, rashes, dermatitis, hyperpigmentation, urticaria, photosensitivity, nail changes, mouth, gum or throat problems, and any toxic side effect caused by a chemotherapy or radiotherapy. Thus, in one preferred embodiment, the agent can be used in combination treatment with a chemotherapeutic agent, where the agent is administered separately, sequentially or concomitantly with the chemotherapeutic agent. Likewise, the agent can be used in combination treatment with radiotherapy, where the agent is administered before, during or after radiotherapy. Another embodiment of the invention relates to an agent as described herein for use in reducing or alleviating one or more chemotherapy-induced or radiotherapy-induced side effects.

In some embodiments, the invention provides methods for treating or reducing the likelihood of metastasis comprising administering an agent described herein during an off-chemotherapy or off-radiotherapy time interval or after the chemotherapy or radiotherapy treatment regimen has been completed. Another embodiment of the invention relates to an agent as described herein for use in treating or reducing the likelihood of metastasis.

In one preferred embodiment, the agent can be used in the treatment of chronic or long term chemotherapy-induced or radiotherapy-induced side effects. Certain toxic effects can appear long after treatment and can result from damage to an organ or system by the therapy. Organ dysfunction, for example, neurological, pulmonary, cardiovascular, and endocrine dysfunction, can be observed in subjects who were treated for cancers during childhood. Chronic or late toxic side effects that occur in subjects who received chemotherapy or radiation therapy include, for example, cardiomyopathy, congestive heart disease, inflammation, early menopause, osteoporosis, infertility, impaired cognitive function, peripheral neuropathy, secondary cancers, cataracts and other vision problems, hearing loss, chronic fatigue, reduced lung capacity, and lung disease.

### Cardiovascular Disease

In one preferred embodiment of the invention, the senescence-associated disease or disorder is a cardiovascular disease. Examples include, but are not limited to, atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm and stroke.

In one preferred embodiment, the senescence-associated disease or disorder is associated with or caused by atherosclerosis (i.e. hardening of the arteries). Atherosclerosis is characterized by patchy intimal plaques (atheromas) that encroach on the lumen of medium-sized and large arteries. Administration of an agent according to the invention can reduce the lipid content of an atherosclerotic plaque in a blood vessel of the subject and/or increase the fibrous cap thickness.

### Inflammatory and Autoimmune Diseases and Disorders

In one preferred embodiment of the invention, the senescence-associated disease or disorder is an inflammatory or autoimmune disease or disorder. Non-limiting examples of autoimmune diseases include osteoporosis, osteoarthritis, psoriasis, oral mucositis, rheumatoid arthritis, inflammatory bowel disease, eczema, kyphosis (curvature of the spinal column), herniated intervertebral disc, and the pulmonary diseases, COPD and idiopathic pulmonary fibrosis. In one preferred embodiment, the senescence-associated disease or disorder is chronic inflammation.

Chronic inflammation is thought to be the main age-related factor associated with osteoarthritis. In combination with aging, joint overuse and obesity appear to promote osteoarthritis. By selectively killing senescent cell, the agents of the invention can reduce or inhibit loss or erosion of proteoglycan layers in a joint, reduce inflammation in the affected joint, and promote production of collagen. Removal of senescent cells causes a reduction in the amount of inflammatory cytokines, such as IL-6, produced in a joint and inflammation is reduced.

In one preferred embodiment of the invention, the senescence-associated disease or disorder is rheumatoid arthritis. Rheumatoid arthritis is a chronic inflammatory disorder that typically affects the joints in hands and feet.

In one preferred embodiment of the invention, the senescence-associated disease or disorder is osteoporosis. Osteoporosis is a progressive bone disease that is characterized by a decrease in bone mass and density that may lead to an increased risk of fracture. Bone mineral density (BMD) is reduced, bone microarchitecture deteriorates, and the amount and variety of proteins in bone are altered.

In one preferred embodiment of the invention, the agent of the invention is for use in treating herniated intervertebral discs. Subjects with herniated discs exhibit elevated presence of cell senescence in the blood and in vessel walls (Roberts et al. (2006) Eur. Spine J. 15 Suppl 3: S312-316).

### Neurological Disease or Disorder

In one preferred embodiment of the invention, the senescence-associated disease or disorder is a neurological disease or disorder selected from Alzheimer's disease (and other dementias), Parkinson's disease, Huntington's disease, dementia, mild cognitive impairment (MCI), macular degeneration and motor neuron dysfunction (MND), and diseases and disorders of the eyes, such as age-related macular degeneration.

Parkinson's disease (PD) is a disabling condition of the brain characterized by slowness of movement (bradykinesia), shaking, stiffness, postural instability and loss of balance. Many of these symptoms are due to the loss of certain nerves in the brain, which results in a lack of dopamine. Senescence of dopamine-producing neurons is thought to contribute to the observed cell death in PD through the production of reactive oxygen species (Cohen et al, J. Neural Transm. Suppl. 19:89-103 (1983)).

Alzheimer's disease (AD) is a neurodegenerative disease that shows a slow progressive mental deterioration with failure of memory, disorientation, and confusion, leading to profound dementia. As the disease progresses, impaired judgment, confusion, behavioral changes, disorientation, and difficulty in walking and swallowing occur. Age is the single greatest predisposing risk factor for developing AD, which is the leading cause of dementia in the elderly (Hebert, et al., Arch. Neurol. 60:1119-1122 (2003)). Early clinical symptoms show remarkable similarity to mild cognitive impairment.

Mild Cognitive Impairment (MCI) is a brain-function syndrome involving the onset and evolution of cognitive impairments beyond those expected based on age and education of the individual, but which are not significant enough to interfere with the individual's daily activities. MCI is an aspect of cognitive aging that is considered to be a transitional state between normal aging and the dementia into which it may convert (Pepeu, Dialogues in Clinical Neuroscience 6:369-377, 2004). MCI that primarily affects memory is known as “amnestic MCI”, which is frequently seen as prodromal stage of Alzheimer's disease. MCI that affects thinking skills other than memory is known as “non-amnestic MCI.”

MND is a group of progressive neurological disorders that destroy motor neurons, the cells that control voluntary muscle activities such as speaking, walking, breathing and swallowing. Examples of MNDs include Amyotrophic Lateral Sclerosis (ALS), also known as Lou Gehrig's Disease, progressive bulbar palsy, pseudobulbar palsy, primary lateral sclerosis, progressive muscular atrophy, lower motor neuron disease, and spinal muscular atrophy (SMA) (e.g., SMA1 also called Werdnig-Hoffmann Disease, SMA2, SMA3 also called Kugelberg-Welander Disease, and Kennedy's disease), post-polio syndrome, and hereditary spastic paraplegia.

### Ophthalmic Diseases and Disorders

In one preferred embodiment of the invention, the senescence-associated disease or disorder is an ocular disease, disorder, or condition. Examples include, but are not limited to, presbyopia, macular degeneration, cataracts and glaucoma. Macular degeneration is a neurodegenerative disease that causes the loss of photoreceptor cells in the central part of retina, called the macula.

### Metabolic Disease

In one preferred embodiment of the invention, the senescence-associated disease or disorder is a metabolic disease selected from diabetes, diabetic ulcer, metabolic syndrome, and obesity.

Senescent cells are understood to play a role in metabolic diseases, such as obesity and type 2 diabetes. Studies have shown that fat tissue from obese mice showed induction of the senescence markers SA-β-Gal, p53, and p21 (Tchkonia et al, Aging Cell 9:667-684 (2010); Minamino et al, Nat. Med. 15: 1082-1087 (2009)). Induction of senescent cells in obesity has potential clinical implications because pro-inflammatory SASP components are also believed to contribute to type 2 diabetes. A similar pattern of up-regulation of senescence markers and SASP components are associated with diabetes, both in mice and in humans. Accordingly, the agents described herein have potential applications in treating or preventing type 2 diabetes, obesity and metabolic syndrome.

### Pulmonary Disease or Disorder

In one preferred embodiment of the invention, the senescence-associated disease or disorder is a pulmonary disease. Examples include, but are not limited to, pulmonary fibrosis, chronic obstructive pulmonary disease (COPD), asthma, cystic fibrosis, emphysema, bronchiectasis, and age-related loss of pulmonary function.

COPD is a lung disease defined by persistently poor airflow resulting from the breakdown of lung tissue (emphysema) and the dysfunction of the small airways (obstructive bronchiolitis). Pulmonary fibrosis is a chronic and progressive lung disease characterized by stiffening and scarring of the lung, which may lead to respiratory failure, lung cancer, and heart failure. The agents of the invention can also be used for treating a subject who is aging and has loss (or degeneration) of pulmonary function (i.e., declining or impaired pulmonary function compared with a younger subject) and/or degeneration of pulmonary tissue.

### Other Age Related Disorders

In one preferred embodiment of the invention, the senescence-associated disease or disorder is an age-related disorder selected from renal disease, renal failure, frailty, hearing loss, muscle fatigue, skin conditions, skin wound healing, liver fibrosis, pancreatic fibrosis, oral submucosa fibrosis, and sarcopenia.

### Dermatological Diseases or Disorders

In one preferred embodiment of the invention, the senescence-associated disease or disorder is a dermatological disease or disorder, Examples include, but are not limited to, eczema, psoriasis, hyperpigmentation, nevi, rashes, atopic dermatitis (a form of eczema and associated with inflammation), urticaria, diseases and disorders related to photosensitivity or photoaging, rhytides (wrinkles due to aging); pruritis (linked to diabetes and aging); dysesthesia (chemotherapy side effect linked to diabetes and multiple sclerosis); eczematous eruptions (often observed in aging patients and linked to side effects of certain drugs); eosinophilic dermatosis (linked to certain kinds of hemotologic cancers); reactive neutrophilic dermatosis (associated with underlying diseases such as inflammatory bowel syndrome); pemphigus; pemphigoid; immunobullous dermatosis (autoimmune blistering of skin); fibrohistocytic proliferations of skin; cutaneous lymphomas; and cutaneous lupus. Late onset lupus may be linked to decreased (i.e., reduced) function of T-cell and B-cells and cytokines (immunosenescence) associated with aging.

### Lifespan and Age-Related Diseases or Conditions

In one preferred embodiment, the agent described herein can be used to extend the lifespan of a subject by selectively killing senescent cells over non-senescent cells. In some embodiments, extending the lifespan of the subject comprises delaying onset or progression of an age-related disease or condition. In some embodiments, the age-related disease or condition is selected from atherosclerosis, cardiovascular disease, cancer, arthritis, dementia, cataract, osteoporosis, diabetes, hypertension, age-related fat loss, lipodystrophy, and kidney disease. In some embodiments, the age-related disease or condition is geriatric anxiety disorder. In some embodiments, the age-related disease or condition is age-related inactivity. In some embodiments, the age-related disease or condition is reduction of spontaneous activity. In some embodiments, the age-related disease or condition is reduction of exploratory behavior.

In one preferred embodiment, the onset or progression of an age-related disease or condition can be delayed by at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year. In one preferred embodiment, the agent is for use in improving the physical health of a subject. In another preferred embodiment, the agent is for use in reverting ageing-associated changes of metabolites in the blood.

Diseases Associated with Elevated β-Galactosidase Activity

In another embodiment, the agent can be used in the treatment of a disease or disorder which correlates, or is associated with, elevated β-galactosidase activity. In a preferred embodiment, the elevated β-galactosidase activity is a result of elevated expression of β-galactosidase or overexpression of β-galactosidase activity relative to baseline levels which can be determined by standard methods.

Expression of β-galactosidase can be detected in cells by histochemical or immunohistochemcal methods. For example, the senescence marker SA-beta galactosidase (SA-β-Gal) can be detected by known methods (Dimri et al, Proc. Natl. Acad. Sci. USA 92: 9363-9367 (1995).

In a more preferred embodiment of the invention, the disease is selected from the Wiedemann-Rautenstrauch syndrome of neonatal progeria, the Werner syndrome of adult progeria, Hutchinson-Gilford syndrome, Rothmund Thompson syndrome, Mulvill-Smith syndrome, Cockayne syndrome, Dyskeratosis Congenita, idiopathic pulmonary fibrosis, aplastic anaemia, emphysema, type 2 diabetes, and degeneration of cartilage.

### Pharmaceutical Compositions

For use according to the present invention, the agents described herein may be presented as a pharmaceutical formulation with one or more pharmaceutically acceptable carriers therefore and optionally other therapeutic and/or prophylactic ingredients. The carrier(s) must be acceptable in the sense of being compatible with the other ingredients of the formulation and not deleterious to the recipient thereof. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller. Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s), buffer(s), flavouring agent(s), surface active agent(s), thickener(s), preservative(s) (including anti-oxidants) and the like, and substances included for the purpose of rendering the formulation isotonic with the blood of the intended recipient.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like. Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation.

The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An agent may also be formulated as dispersible granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g. tablets wherein an agent is formulated in an appropriate release-controlling matrix, or is coated with a suitable release-controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds. Pharmaceutical formulations suitable for parenteral administration include sterile solutions or suspensions of an active compound in aqueous or oleaginous vehicles.

Injectable preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An agent may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility, such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof. As a further possibility, an agent may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

Pharmaceutical or veterinary composition as described above can be prepared by bringing the agent into association with the carrier, for example by admixture.

In general, the formulations are prepared by uniformly and intimately bringing into association the agent with liquid carriers or finely divided solid carriers or both, and then if necessary shaping the product.

### Salts

The agents of the invention can be present as salts, in particular pharmaceutically and veterinarily acceptable salts.

Pharmaceutically acceptable salts of the agents of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. hydrohalic acids such as hydrochloride, hydrobromide and hydroiodide, sulphuric acid, phosphoric acid sulphate, bisulphate, hemisulphate, thiocyanate, persulphate and sulphonic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Salts which are not pharmaceutically or veterinarily acceptable may still be valuable as intermediates.

Preferred salts include, for example, acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids.

### Enantiomers/Tautomers

In all aspects of the present invention previously discussed, the invention includes, where appropriate all enantiomers, diastereoisomers and tautomers of the agents of the invention. The person skilled in the art will recognise compounds that possess optical properties (one or more chiral carbon atoms) or tautomeric characteristics. The corresponding enantiomers and/or tautomers may be isolated/prepared by methods known in the art.

Enantiomers are characterised by the absolute configuration of their chiral centres and described by the R- and S-sequencing rules of Cahn, Ingold and Prelog. Such conventions are well known in the art (e.g. see ‘Advanced Organic Chemistry’, 3^{rd} edition, ed. March, J., John Wiley and Sons, New York, 1985).

Agents of the invention containing a chiral centre may be used as a racemic mixture, an enantiomerically enriched mixture, or the racemic mixture may be separated using well-known techniques and an individual enantiomer may be used alone.

### Stereo and Geometric Isomers

Some of the agents for use in the invention may exist as stereoisomers and/or geometric isomers, e.g. they may possess one or more asymmetric and/or geometric centres and so may exist in two or more stereoisomeric and/or geometric forms. The present invention contemplates the use of all the individual stereoisomers and geometric isomers of those agents, and mixtures thereof. The terms used in the claims encompass these forms, provided said forms retain the appropriate functional activity (though not necessarily to the same degree).

The present invention also includes all suitable isotopic variations of the agents. An isotopic variation is defined as one in which at least one atom is replaced by an atom having the same atomic number but an atomic mass different from the atomic mass usually found in nature. Examples of isotopes that can be incorporated include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸, ³¹P, ³²P, ³⁵S, ¹⁸F and ³⁶Cl, respectively. Certain isotopic variations, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. For example, the invention includes agents where any hydrogen atom has been replaced by a deuterium atom. Isotopic variations of the agent of the present invention and pharmaceutically acceptable salts thereof of this invention can generally be prepared by conventional procedures using appropriate isotopic variations of suitable reagents.

### Solvates

The present invention also includes solvate forms of the agents of the present invention. The terms used in the claims encompass these forms.

### Polymorphs

The invention further relates to the agents of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Administration

The pharmaceutical compositions of the present invention may be adapted for rectal, nasal, intrabronchial, topical (including buccal and sublingual), vaginal or parenteral (including subcutaneous, intramuscular, intravenous, intraarterial and intradermal), intraperitoneal or intrathecal administration. Preferably the formulation is an orally administered formulation. The formulations may conveniently be presented in unit dosage form, i.e., in the form of discrete portions containing a unit dose, or a multiple or sub-unit of a unit dose. By way of example, the formulations may be in the form of tablets and sustained release capsules, and may be prepared by any method well known in the art of pharmacy.

Formulations for oral administration in the present invention may be presented as: discrete units such as capsules, gellules, drops, cachets, pills or tablets each containing a predetermined amount of the active agent; as a powder or granules; as a solution, emulsion or a suspension of the active agent in an aqueous liquid or a non-aqueous liquid; or as an oil-in-water liquid emulsion or a water-in-oil liquid emulsion; or as a bolus etc. Preferably, these compositions contain from 1 to 250 mg and more preferably from 10-100 mg, of active ingredient per dose.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropyl-methylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free-flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Other forms of administration comprise solutions or emulsions which may be injected intravenously, intraarterially, intrathecally, subcutaneously, intradermally, intraperitoneally or intramuscularly, and which are prepared from sterile or sterilisable solutions. Injectable forms typically contain between 10-1000 mg, preferably between 10-250 mg, of active ingredient per dose.

The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

The agents of this invention may also be administered orally to the patient, in a manner such that the concentration of drug is sufficient to achieve one or more of the therapeutic indications disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when agents of the present invention are administered in accordance with the present invention. The agents of this invention, which may have good bioavailability, may be tested in one of several biological assays to determine the concentration of a compound which is required to have a given pharmacological effect.

### Combinations

In a particularly preferred embodiment, the agents of the invention are administered in combination with one or more other active agents, for example, existing drugs available on the market. In such cases, the compounds of the invention may be administered consecutively, simultaneously or sequentially with the one or more other active agents. Drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease or delay the emergence of resistance. Beneficial combinations may be suggested by studying the activity of the test compounds with agents known or suspected of being valuable in the treatment of a particular disorder.

The invention is further described by way of the following non-limiting examples.

### Examples

### Materials and Methods

The agents described herein are commercially available. For example, the following compounds were obtained from The Library of Pharmacologically Active Compounds (LOPAC; Sigma Aldrich):

### PF-573228

### JFD00244

### Ouabain

### Bufalin

### K-Strophanthin

### Strophanthidin

### Digoxin

### Cyclosporine

### Tyrphostin AG879

### Cantharidin

Diphenyleneiodonium chloride (DPI)

### Rottlerin

2,3-Dimethoxy-1,4-naphthoquinone

### LY-367,265

### Rotenone

### Idarubicin

Dequalinium chloride

### Vincristine

The following compounds were obtained from the Selleck chemical library:

Atorvastatin calcium (Lipitor)
Fluvastatin sodium (Lescol)

### Lovastatin (Mevacor)

Pitavastatin calcium (Livalo)

### Simvastatin (Zocor)

### Nitazoxanide (Alinia, Annita)

### Nitrofurazone (Nitrofural)

### Cyclosporine (Neoral)

### Temsirolimus (Torisel)

### Eltrombopag (SB-497115-GR)

### Vincristine

### Adapalene

### Azacyclonol

### Enoxacin (Penetrex)

### Raltegravir (MK-0518).

Other FAK inhibitors (compound 14 also known as NSC 677249 and Defactinib) and other chemical compounds (such as etoposide and doxorubicin) were obtained from SIGMA.

IMR90 cells were obtained from ATCC. IMR90 ER:RAS and IMR90 ER:RAS E6/E7 were generated by retroviral infection of IMR90 cells and have been described elsewhere (Banito et al., 2009; Barradas et al., 2009). HMEC cells were obtained from Lonza. HMEC hTERT ER:RAS were generated by retroviral infaction of HMEC cells. (PF-573228, compound 14 also known as NSC 677249 and Defactinib)

### Determining Senolytic Activity

IMR90 ER:RAS cells were induced to undergo senescence by treating them with 100 nM 4OHT for 6 days. At that point, 10 μM of the indicated drugs was added. In parallel, the same treatments were carried out in IMR90 ER:RAS cells treated with DMSO (−4OHT). These cells do not undergo senescence. Cells were fixed at day 6, 8 and 10 after 4OHT induction and stained with crystal violet to assess cell viability. A similar experiment was conducted and cells at day 10 post-induction were fixed and stained with DAPI to assess cell numbers (see FIG. 1). Different models of senescence were used to test the senolytic compounds in cell culture (see FIG. 2).

Candidate senolytic agents were tested for their ability:
To kill cells undergoing oncogene-induced senescence (see FIG. 3);
To kill HMEC cells undergoing oncogene-induced senescence (see FIG. 4);
To kill IMR90 ER:RAS E6/E7 cells (see FIG. 5);
To kill old IMR90 cells (see FIG. 6).
To kill IMR90 cells undergoing senescence by Etoposide treatment (see FIG. 7).
To kill IMR90 cells undergoing senescence by Doxorubicin treatment (see FIG. 8);
To kill cells undergoing oncogene-induced senescence (see FIG. 10)

In each instance, the methodology used was in accordance with that set forth above. The results are summarised in FIG. 9.

The results indicated that statins behave as senolytics, preferentially killing IMR90 ER:RAS cells that undergo oncogene-induced senescence (see FIG. 11).

The results also indicated that Focal adhesion kinase (FAK) inhibitors behave as senolytics. Three different FAK inhibitors (PF-573228, compound 14 also known as NSC 677249 and Defactinib) were tested on IMR90 ER:RAS cells to analyse their ability to kill differentially senescent cells (4OHT induced) vs non-senescent cells (DMSO treated). These compounds were shown to preferentially kill senescent cells in a dose-dependent manner (see FIG. 12).

Several of the agents described herein kill senescent cells by inducing apoptosis. IMR90 ER:RAS cells were treated with DMSO (as a control) or 4OHT (to induce senescence). Consequently, cells were treated with DMSO, ABT-263 or the indicated senolytics for 2 (A) or 3 days (B) in the presence or absence of the pan-caspase inhibitor QVD. Treatment of senescent cells with senolytics kills them, but is prevented in the case of PF-573228, rottlerin and rotenone by pre-treatment with the pan-caspase inhibitor QVD (see FIG. 13).

Compounds JFD 00244 and Rottlerin specifically eliminate senescent cells in mixed cultures containing normal and senescent cells (see FIG. 14). (A) Setup of the experiments, in which normal cells (IMR90 Cherry) are mixed with IMR90 ER:RAS GFP cells (which become senescence in the presence of 4OHT). (B) Representative pictures showing the mixed cultures treated with the indicated drugs. In the bottom row, upon 4OHT the IMR90 ER:RAS GFP cells are senescent. (C) Quantification of the IMR90 Cherry and IMR90 ER:RAS GFP in the mixed cultures.

Ouabain eliminates senescent cells induced by Doxorubicin treatment in mice (see FIG. 15). (A) Experimental set up, mice (n=5 per group) were administered with a single dose of 10 mg/kg of Doxorubicin. 2 months later mice were treated with vehicle, Ouabain or ABT-263 (as a senolytic control) for 4 consecutive days, before being culled for analysis. (B) Cryosections of lungs obtained from control and doxorubicin treated mice were stained with hematoxilin and eosin (to reveal tissue architecture), and SA-β-Gal (to reveal senescent cells). Senescent cells accumulate in the bronchioles of doxorubicin-treated mice. (C) Quantification of the lung area positive for SA-β-Gal staining. Treatment with doxorubicin induces the accumulation of senescent cells that can be partially eliminated upon treatment with ABT-263 or Ouabain. (D) Representative pictures of lung cryosections.

Treatment with DPI or Ouabain eliminates senescent cells induced by whole body irradiation in mice (see FIG. 16). (A) Experimental set up, mice (n=5 per group) were irradiated with 6 Gray to induce senescence. 2 months later mice were treated with vehicle, DPI, Ouabain or ABT-263 (as a senolytic control) for 4 consecutive days, before being culled for analysis. (B) Cryosections of lungs obtained from control and irradiated mice were stained with hematoxilin and eosin (to reveal tissue architecture), and SA-β-Gal (to reveal senescent cells). Senescent cells accumulate in the bronchioles of irradiated mice. (C) Quantification of the lung area positive for SA-β-Gal staining. Whole body irradiation induces the accumulation of senescent cells that can be partially eliminated upon treatment with ABT-263, Ouabain or DPI. (D). Representative pictures of lung cryosections.

Further embodiments of the invention are set forth in the following numbered paragraphs:

1. An agent for use in selectively killing one or more senescent cells, wherein said agent is selected from the following: a focal adhesion kinase (FAK) inhibitor, an HMG-CoA reductase inhibitor, JFD00244, Ouabain, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Nitazoxanide, Nitrofurazone, Temsirolimus, Eltrombopag, Adapalene, Azacyclonol, Enoxacin and Raltegravir, and pharmaceutically acceptable salts thereof.
2. An agent for use according to paragraph 1 which is an HMG-CoA reductase inhibitor.
3. An agent for use according to paragraph 2 wherein the HMG-CoA reductase inhibitor is a statin.
4. An agent for use according to paragraph 3 wherein the statin is selected from Atorvastatin calcium, Fluvastatin sodium, Lovastatin, Pitavastatin calcium and Simvastatin.
5. An agent for use according to paragraph 1 which is a focal adhesion kinase (FAK) inhibitor.
6. An agent for use according to paragraph 5, wherein the focal adhesion kinase (FAK) inhibitor is selected from PF-573228, NSC 677249 and Defactinib.
7. A method of selectively killing one or more senescent cells in a subject, said method comprising administering to the subject a therapeutically effective amount of an agent selected from the following: a focal adhesion kinase (FAK) inhibitor, an HMG-CoA reductase inhibitor, JFD00244, Ouabain, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Nitazoxanide, Nitrofurazone, Temsirolimus, Eltrombopag, Adapalene, Azacyclonol, Enoxacin and Raltegravir, and pharmaceutically acceptable salts thereof.
8. A compound for use in treating or preventing a senescence-associated disease or disorder, wherein said compound is an HMG-CoA reductase inhibitor, and said senescence-associated disease or disorder is other than a cardiovascular disorder.
9. A compound for use according to paragraph 8, wherein the HMG-CoA reductase inhibitor is a statin.
10. A compound for use according to paragraph 9 wherein the statin is selected from Atorvastatin calcium, Fluvastatin sodium, Lovastatin, Pitavastatin calcium and Simvastatin.
11. A compound for use according to any one of paragraphs 8 to 10, wherein the senescence-associated disease or disorder is a metabolic disease, an inflammatory disease or disorder, a pulmonary disease or disorder, a neurological disease or disorder, a proliferative disorder, a renal disorder or disease, an eye disease or disorder, or a dermatological disorder or disease.
12. A compound for use according to any one of paragraphs 8 to 11 wherein the senescence-associated disease or disorder is selected from:
(i) an inflammatory or autoimmune disease or disorder selected from osteoarthritis, osteoporosis, oral mucositis, inflammatory bowel disease, kyphosis and herniated intervertebral disc;
(ii) a neurological disease or disorder selected from Alzheimer's disease, Parkinson's disease, Huntington's disease, dementia, mild cognitive impairment, macular degeneration and motor neuron dysfunction;
(iii) a metabolic disease selected from diabetes, diabetic ulcer, metabolic syndrome and obesity;
(iv) a pulmonary disease selected from pulmonary fibrosis, chronic obstructive pulmonary disease, asthma, cystic fibrosis, emphysema, bronchiectasis and age-related loss of pulmonary function;
(v) an eye disease or disorder selected from macular degeneration, glaucoma, cataracts, presbyopia and vision loss;
(vi) an age-related disorder selected from renal disease, renal failure, frailty, hearing loss, muscle fatigue, skin conditions, skin wound healing, liver fibrosis, pancreatic fibrosis, oral submucosa fibrosis and sarcopenia; and
(vii) a dermatological disease or disorder selected from eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas and cutaneous lupus.
13. A compound for use in treating or preventing a senescence-associated disease or disorder, wherein said compound is a focal adhesion kinase (FAK) inhibitor.
14. A compound for use according to paragraph 13 wherein the focal adhesion kinase (FAK) inhibitor is selected from PF-573228, NSC 677249 and Defactinib.
15. A compound for use according to paragraph 13 or paragraph 14, wherein the senescence-associated disease or disorder is a cardiovascular disease or disorder, a metabolic disease, an inflammatory disease or disorder, a pulmonary disease or disorder, a neurological disease or disorder, a renal disorder or disease, or a dermatological disorder or disease.
16. A compound for use according to any one of paragraphs 13 to 15 wherein the senescence-associated disease or disorder is selected from:
(i) an inflammatory or autoimmune disease or disorder selected from osteoarthritis, osteoporosis, oral mucositis, inflammatory bowel disease, kyphosis and herniated intervertebral disc;
(ii) a neurological disease or disorder selected from Alzheimer's disease, Parkinson's disease, Huntington's disease, dementia, mild cognitive impairment, macular degeneration and motor neuron dysfunction;
(iii) a metabolic disease selected from diabetes, diabetic ulcer, metabolic syndrome and obesity;
(iv) a pulmonary disease selected from pulmonary fibrosis, chronic obstructive pulmonary disease, asthma, cystic fibrosis, emphysema, bronchiectasis and age-related loss of pulmonary function;
(v) an eye disease or disorder selected from macular degeneration, glaucoma, cataracts, presbyopia and vision loss;
(vi) an age-related disorder selected from renal disease, renal failure, frailty, hearing loss, muscle fatigue, skin conditions, skin wound healing, liver fibrosis, pancreatic fibrosis, oral submucosa fibrosis and sarcopenia;
(vii) a dermatological disease or disorder selected from eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas, and cutaneous lupus; and
(viii) a cardiovascular disease selected from atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapse, peripheral vascular disease, cardiac stress resistance, cardiac fibrosis, brain aneurysm and stroke.
17. A compound for use in treating or preventing a senescence-associated disease or disorder, wherein said compound is selected from the following: JFD00244, Cyclosporine, Tyrphostin AG879, Cantharidin, Diphenyleneiodonium chloride, 2,3-Dimethoxy-1,4-naphthoquinone, LY-367,265, Rotenone, Idarubicin, Dequalinium chloride, Vincristine, Atorvastatin calcium, Nitazoxanide, Nitrofurazone, Temsirolimus, Eltrombopag, Adapalene, Azacyclonol, Enoxacin and Raltegravir, and pharmaceutically acceptable salts thereof, where the compound is not already in salt form.
18. A compound for use according to paragraph 17 wherein the senescence-associated disease or disorder is a cardiovascular disease or disorder, a metabolic disease, an inflammatory disease or disorder, a pulmonary disease or disorder, a neurological disease or disorder, a renal disorder or disease, or a dermatological disorder or disease.
19. A compound for use according to paragraph 17 or paragraph 18 wherein the senescence-associated disease or disorder is selected from:
(i) an inflammatory or autoimmune disease or disorder selected from osteoarthritis, osteoporosis, oral mucositis, inflammatory bowel disease, kyphosis and herniated intervertebral disc;
(ii) a neurological disease or disorder selected from Alzheimer's disease, Parkinson's disease, Huntington's disease, dementia, mild cognitive impairment, macular degeneration and motor neuron dysfunction;
(iii) a metabolic disease selected from diabetes, diabetic ulcer, metabolic syndrome and obesity;
(iv) a pulmonary disease selected from pulmonary fibrosis, chronic obstructive pulmonary disease, asthma, cystic fibrosis, emphysema, bronchiectasis and age-related loss of pulmonary function;
(v) an eye disease or disorder selected from macular degeneration, glaucoma, cataracts, presbyopia and vision loss;
(vi) an age-related disorder selected from renal disease, renal failure, frailty, hearing loss, muscle fatigue, skin conditions, skin wound healing, liver fibrosis, pancreatic fibrosis, oral submucosa fibrosis and sarcopenia;
(vii) a dermatological disease or disorder selected from eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas and cutaneous lupus; and
(viii) a cardiovascular disease selected from atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapse, peripheral vascular disease, cardiac stress resistance, cardiac fibrosis, brain aneurysm and stroke.
20. A compound for use in treating or preventing a senescence-associated disease or disorder, wherein said senescence-associated disease or disorder is other than a cardiovascular disorder, and wherein said compound is selected from the following: PF-573228, JFD00244, Ouabain, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin and Raltegravir, and pharmaceutically acceptable salts thereof, where the compound is not already in salt form.
21. A compound for use according to paragraph 20 wherein the senescence-associated disease or disorder is a metabolic disease, an inflammatory disease or disorder, a pulmonary disease or disorder, a neurological disease or disorder, a renal disorder or disease, or a dermatological disorder or disease.
22. A compound for use according to paragraph 20 or paragraph 21 wherein the senescence-associated disease or disorder is selected from:
(i) an inflammatory or autoimmune disease or disorder selected from osteoarthritis, osteoporosis, oral mucositis, inflammatory bowel disease, kyphosis and herniated intervertebral disc;
(ii) a neurological disease or disorder selected from Alzheimer's disease, Parkinson's disease, Huntington's disease, dementia, mild cognitive impairment, macular degeneration and motor neuron dysfunction;
(iii) a metabolic disease selected from diabetes, diabetic ulcer, metabolic syndrome and obesity;
(iv) a pulmonary disease selected from pulmonary fibrosis, chronic obstructive pulmonary disease, asthma, cystic fibrosis, emphysema, bronchiectasis and age-related loss of pulmonary function;
(v) an eye disease or disorder selected from macular degeneration, glaucoma, cataracts, presbyopia and vision loss;
(vi) an age-related disorder selected from renal disease, renal failure, frailty, hearing loss, muscle fatigue, skin conditions, skin wound healing, liver fibrosis, pancreatic fibrosis, oral submucosa fibrosis and sarcopenia; and
(vii) a dermatological disease or disorder is selected from eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas and cutaneous lupus.
23. A method for treating or preventing a senescence-associated disease or disorder other than a cardiovascular disorder, said method comprising administering to a subject a therapeutically effective amount of an HMG-CoA reductase inhibitor.
24. A method for treating or preventing a senescence-associated disease or disorder, said method comprising administering to a subject a therapeutically effective amount of a focal adhesion kinase (FAK) inhibitor.
25. A method for treating or preventing a senescence-associated disease or disorder by selectively killing senescent cells, said method comprising administering to a subject a therapeutically effective amount of a compound selected from the following: PF-573228, JFD00244, Ouabain, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin, Raltegravir, NSC 677249 and Defactinib, and pharmaceutically acceptable salts thereof, where the compound is not already in salt form.
26. Use of a compound selected from the following: PF-573228, JFD00244, Ouabain, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin, Raltegravir, NSC 677249 and Defactinib, and pharmaceutically acceptable salts thereof, where the compound is not already in salt form;
in the preparation of a medicament for treating or preventing a senescence-associated disease or disorder, wherein said compound selectively kills one or more senescent cells.
27. Use of a focal adhesion kinase (FAK) inhibitor in the preparation of a medicament for treating or preventing a senescence-associated disease or disorder.
28. Use of an HMG-CoA reductase inhibitor in the preparation of a medicament for treating or preventing a senescence-associated disease or disorder, wherein said senescence-associated disease or disorder is other than a cardiovascular disorder.
29. A senolytic agent which is selected from the following: PF-573228, JFD00244, Ouabain, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin, Raltegravir, NSC 677249 and Defactinib, and pharmaceutically acceptable salts thereof, where the compound is not already in salt form.
30. A compound selected from the following: PF-573228, JFD00244, Ouabain, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin, Raltegravir, NSC 677249 and Defactinib, and pharmaceutically acceptable salts thereof, where the compound is not already in salt form; for use in extending the lifespan of a subject, preferably by delaying onset or progression of an age-related disease or condition.
31. A compound selected from the following: PF-573228, JFD00244, Ouabain, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin and Raltegravir, and pharmaceutically acceptable salts thereof, where the compound is not already in salt form; for use in treating cancer, and wherein said compound:
reduces or eliminates eliminate cancer cells that have been pushed to senescence; or
reduces or eliminates one or more side effects produced by senescent cells, wherein said side effects comprise inflammation, promotion of cancer growth and promotion of metastasis; or
reduces or eliminates one or more side effects of chemotherapy; or
reduces or eliminates one or more side side effects or radiotherapy; or
reduces or eliminates precancerous lesions; or
reduces or eliminates cells that undergo senescence by treatment with CDK4 or CDK6 inhibitors.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

### REFERENCES

Baker, D. J., Childs, B. G., Durik, M., Wijers, M. E., Sieben, C. J., Zhong, J., Saltness, R. A., Jeganathan, K. B., Verzosa, G. C., Pezeshki, A., et al. (2016). Naturally occurring p16(Ink4a)-positive cells shorten healthy lifespan. Nature 530, 184-189.
Baker, D. J., Wijshake, T., Tchkonia, T., LeBrasseur, N. K., Childs, B. G., van de Sluis, B., Kirkland, J. L., and van Deursen, J. M. (2011). Clearance of p16Ink4a-positive senescent cells delays ageing-associated disorders. Nature 479, 232-236.
Banito, A., Rashid, S. T., Acosta, J. C., Li, S., Pereira, C. F., Geti, I., Pinho, S., Silva, J. C., Azuara, V., Walsh, M., et al. (2009). Senescence impairs successful reprogramming to pluripotent stem cells. Genes Dev 23, 2134-2139.
Barradas, M., Anderton, E., Acosta, J. C., Li, S., Banito, A., Rodriguez-Niedenfuhr, M., Maertens, G., Banck, M., Zhou, M. M., Walsh, M. J., et al. (2009). Histone demethylase JMJD3 contributes to epigenetic control of INK4a/ARF by oncogenic RAS. Genes Dev 23, 1177-1182.
Chang, J., Wang, Y., Shao, L., Laberge, R. M., Demaria, M., Campisi, J., Janakiraman, K., Sharpless, N. E., Ding, S., Feng, W., et al. (2016). Clearance of senescent cells by ABT263 rejuvenates aged hematopoietic stem cells in mice. Nat Med 22, 78-83.
Childs, B. G., Baker, D. J., Wijshake, T., Conover, C. A., Campisi, J., and van Deursen, J. M. (2016). Senescent intimal foam cells are deleterious at all stages of atherosclerosis. Science 354, 472-477.
Demaria, M., O'Leary, M. N., Chang, J., Shao, L., Liu, S., Alimirah, F., Koenig, K., Le, C., Mitin, N., Deal, A. M., et al. (2016). Cellular Senescence Promotes Adverse Effects of Chemotherapy and Cancer Relapse. Cancer Discov.
Demaria, M., Ohtani, N., Youssef, S. A., Rodier, F., Toussaint, W., Mitchell, J. R., Laberge, R. M., Vijg, J., Van Steeg, H., Dolle, M. E., et al. (2014). An essential role for senescent cells in optimal wound healing through secretion of PDGF-AA. Dev Cell 31, 722-733.
Gerber, R., Ryan, J. D., and Clark, D. S. (2004). Cell-based screen of HMG-CoA reductase inhibitors and expression regulators using LC-MS. Anal Biochem 329, 28-34.
Goldstein, J. L., and Brown, M. S. (2015). A century of cholesterol and coronaries: from plaques to genes to statins. Cell 161, 161-172.
Kuilman, T., Michaloglou, C., Mooi, W. J., and Peeper, D. S. (2010). The essence of senescence. Genes Dev 24, 2463-2479.
Munoz-Espin, D., and Serrano, M. (2014). Cellular senescence: from physiology to pathology. Nat Rev Mol Cell Biol 15, 482-496.
Wang, Y., Chang, J., Liu, X., Zhang, X., Zhang, S., Zhang, X., Zhou, D., and Zheng, G. (2016). Discovery of piperlongumine as a potential novel lead for the development of senolytic agents. Aging (Albany N.Y.) 8, 2915-2926.
Yosef, R., Pilpel, N., Tokarsky-Amiel, R., Biran, A., Ovadya, Y., Cohen, S., Vadai, E., Dassa, L., Shahar, E., Condiotti, R., et al. (2016). Directed elimination of senescent cells by inhibition of BCL-W and BCL-XL. Nat Commun 7, 11190.
Zhu, Y., Tchkonia, T., Fuhrmann-Stroissnigg, H., Dai, H. M., Ling, Y. Y., Stout, M. B., Pirtskhalava, T., Giorgadze, N., Johnson, K. O., Giles, C. B., et al. (2016). Identification of a novel senolytic agent, navitoclax, targeting the Bcl-2 family of anti-apoptotic factors. Aging Cell 15, 428-435.
Zhu, Y., Tchkonia, T., Pirtskhalava, T., Gower, A. C., Ding, H., Giorgadze, N., Palmer, A. K., Ikeno, Y., Hubbard, G. B., Lenburg, M., et al. (2015). The Achilles' heel of senescent cells: from transcriptome to senolytic drugs. Aging Cell 14, 644-658.

1. An agent for use in selectively killing one or more senescent cells, wherein said agent is selected from the following: a cardiac glycoside or aglycone, a focal adhesion kinase (FAK) inhibitor, an HMG-CoA reductase inhibitor, JFD00244, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Nitazoxanide, Nitrofurazone, Temsirolimus, Eltrombopag, Adapalene, Azacyclonol, Enoxacin and Raltegravir, and pharmaceutically acceptable salts thereof.

2. An agent for use according to claim 1 which is a cardiac glycoside or an aglycone.

3. An agent for use according to claim 2 which is a cardiac glycoside selected from Ouabain, Digoxin, and K-Strophanthin.

4. An agent for use according to claim 2 which is an aglycone selected from Strophanthidin and Bufalin.

5. An agent for use according to claim 1 which is an HMG-CoA reductase inhibitor.

6. An agent for use according to claim 5 wherein the HMG-CoA reductase inhibitor is a statin.

7. An agent for use according to claim 6 wherein the statin is selected from Atorvastatin calcium, Fluvastatin sodium, Lovastatin, Pitavastatin calcium and Simvastatin.

8. An agent for use according to claim 1 which is a focal adhesion kinase (FAK) inhibitor.

9. An agent for use according to claim 8, wherein the focal adhesion kinase (FAK) inhibitor is selected from PF-573228, NSC 677249 and Defactinib.

10. A method of selectively killing one or more senescent cells in a subject, said method comprising administering to the subject a therapeutically effective amount of an agent selected from the following: a cardiac glycoside or aglycone, a focal adhesion kinase (FAK) inhibitor, an HMG-CoA reductase inhibitor, JFD00244, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Nitazoxanide, Nitrofurazone, Temsirolimus, Eltrombopag, Adapalene, Azacyclonol, Enoxacin and Raltegravir, and pharmaceutically acceptable salts thereof.

11. A compound for use in treating or preventing a senescence-associated disease or disorder, wherein said compound is an HMG-CoA reductase inhibitor, and said senescence-associated disease or disorder is other than a cardiovascular disorder.

12. A compound for use according to claim 11, wherein the HMG-CoA reductase inhibitor is a statin.

13. A compound for use according to claim 12 wherein the statin is selected from Atorvastatin calcium, Fluvastatin sodium, Lovastatin, Pitavastatin calcium and Simvastatin.

14. A compound for use according to any one of claims 11 to 13, wherein the senescence-associated disease or disorder is a metabolic disease, an inflammatory disease or disorder, a pulmonary disease or disorder, a neurological disease or disorder, a proliferative disorder, a renal disorder or disease, an eye disease or disorder, or a dermatological disorder or disease.

15. A compound for use according to any one of claims 11 to 14 wherein the senescence-associated disease or disorder is selected from:
(i) an inflammatory or autoimmune disease or disorder selected from osteoarthritis, osteoporosis, oral mucositis, inflammatory bowel disease, kyphosis and herniated intervertebral disc;
(ii) a neurological disease or disorder selected from Alzheimer's disease, Parkinson's disease, Huntington's disease, dementia, mild cognitive impairment, macular degeneration and motor neuron dysfunction;
(iii) a metabolic disease selected from diabetes, diabetic ulcer, metabolic syndrome and obesity;
(iv) a pulmonary disease selected from pulmonary fibrosis, chronic obstructive pulmonary disease, asthma, cystic fibrosis, emphysema, bronchiectasis and age-related loss of pulmonary function;
(v) an eye disease or disorder selected from macular degeneration, glaucoma, cataracts, presbyopia and vision loss;
(vi) an age-related disorder selected from renal disease, renal failure, frailty, hearing loss, muscle fatigue, skin conditions, skin wound healing, liver fibrosis, pancreatic fibrosis, oral submucosa fibrosis and sarcopenia; and
(vii) a dermatological disease or disorder selected from eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas and cutaneous lupus.

16. A compound for use in treating or preventing a senescence-associated disease or disorder, wherein said compound is a focal adhesion kinase (FAK) inhibitor.

17. A compound for use according to claim 16 wherein the focal adhesion kinase (FAK) inhibitor is selected from PF-573228, NSC 677249 and Defactinib.

18. A compound for use according to claim 16 or claim 17, wherein the senescence-associated disease or disorder is a cardiovascular disease or disorder, a metabolic disease, an inflammatory disease or disorder, a pulmonary disease or disorder, a neurological disease or disorder, a renal disorder or disease, or a dermatological disorder or disease.

19. A compound for use according to any one of claims 16 to 18 wherein the senescence-associated disease or disorder is selected from:
(i) an inflammatory or autoimmune disease or disorder selected from osteoarthritis, osteoporosis, oral mucositis, inflammatory bowel disease, kyphosis and herniated intervertebral disc;
(ii) a neurological disease or disorder selected from Alzheimer's disease, Parkinson's disease, Huntington's disease, dementia, mild cognitive impairment, macular degeneration and motor neuron dysfunction;
(iii) a metabolic disease selected from diabetes, diabetic ulcer, metabolic syndrome and obesity;
(iv) a pulmonary disease selected from pulmonary fibrosis, chronic obstructive pulmonary disease, asthma, cystic fibrosis, emphysema, bronchiectasis and age-related loss of pulmonary function;
(v) an eye disease or disorder selected from macular degeneration, glaucoma, cataracts, presbyopia and vision loss;
(vi) an age-related disorder selected from renal disease, renal failure, frailty, hearing loss, muscle fatigue, skin conditions, skin wound healing, liver fibrosis, pancreatic fibrosis, oral submucosa fibrosis and sarcopenia;
(vii) a dermatological disease or disorder selected from eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas, and cutaneous lupus; and
(viii) a cardiovascular disease selected from atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapse, peripheral vascular disease, cardiac stress resistance, cardiac fibrosis, brain aneurysm and stroke.

20. A compound for use in treating or preventing a senescence-associated disease or disorder, wherein said compound is selected from the following: JFD00244, Cyclosporine, Tyrphostin AG879, Cantharidin, Diphenyleneiodonium chloride, 2,3-Dimethoxy-1,4-naphthoquinone, LY-367,265, Rotenone, Idarubicin, Dequalinium chloride, Vincristine, Atorvastatin calcium, Nitazoxanide, Nitrofurazone, Temsirolimus, Eltrombopag, Adapalene, Azacyclonol, Enoxacin and Raltegravir, and pharmaceutically acceptable salts thereof, where the compound is not already in salt form.

21. A compound for use according to claim 20 wherein the senescence-associated disease or disorder is a cardiovascular disease or disorder, a metabolic disease, an inflammatory disease or disorder, a pulmonary disease or disorder, a neurological disease or disorder, a renal disorder or disease, or a dermatological disorder or disease.

22. A compound for use according to claim 20 or claim 21 wherein the senescence-associated disease or disorder is selected from:
(i) an inflammatory or autoimmune disease or disorder selected from osteoarthritis, osteoporosis, oral mucositis, inflammatory bowel disease, kyphosis and herniated intervertebral disc;
(ii) a neurological disease or disorder selected from Alzheimer's disease, Parkinson's disease, Huntington's disease, dementia, mild cognitive impairment, macular degeneration and motor neuron dysfunction;
(iii) a metabolic disease selected from diabetes, diabetic ulcer, metabolic syndrome and obesity;
(iv) a pulmonary disease selected from pulmonary fibrosis, chronic obstructive pulmonary disease, asthma, cystic fibrosis, emphysema, bronchiectasis and age-related loss of pulmonary function;
(v) an eye disease or disorder selected from macular degeneration, glaucoma, cataracts, presbyopia and vision loss;
(vi) an age-related disorder selected from renal disease, renal failure, frailty, hearing loss, muscle fatigue, skin conditions, skin wound healing, liver fibrosis, pancreatic fibrosis, oral submucosa fibrosis and sarcopenia;
(vii) a dermatological disease or disorder selected from eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas and cutaneous lupus; and
(viii) a cardiovascular disease selected from atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapse, peripheral vascular disease, cardiac stress resistance, cardiac fibrosis, brain aneurysm and stroke.

23. A compound for use in treating or preventing a senescence-associated disease or disorder, wherein said senescence-associated disease or disorder is other than a cardiovascular disorder, and wherein said compound is selected from the following: PF-573228, JFD00244, Ouabain, Bufalin, Digoxin, K-Strophanthin, Strophanthidin, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin and Raltegravir, and pharmaceutically acceptable salts thereof, where the compound is not already in salt form.

24. A compound for use according to claim 23 wherein the senescence-associated disease or disorder is a metabolic disease, an inflammatory disease or disorder, a pulmonary disease or disorder, a neurological disease or disorder, a renal disorder or disease, or a dermatological disorder or disease.

25. A compound for use according to claim 23 or claim 24 wherein the senescence-associated disease or disorder is selected from:
(i) an inflammatory or autoimmune disease or disorder selected from osteoarthritis, osteoporosis, oral mucositis, inflammatory bowel disease, kyphosis and herniated intervertebral disc;
(ii) a neurological disease or disorder selected from Alzheimer's disease, Parkinson's disease, Huntington's disease, dementia, mild cognitive impairment, macular degeneration and motor neuron dysfunction;
(iii) a metabolic disease selected from diabetes, diabetic ulcer, metabolic syndrome and obesity;
(iv) a pulmonary disease selected from pulmonary fibrosis, chronic obstructive pulmonary disease, asthma, cystic fibrosis, emphysema, bronchiectasis and age-related loss of pulmonary function;
(v) an eye disease or disorder selected from macular degeneration, glaucoma, cataracts, presbyopia and vision loss;
(vi) an age-related disorder selected from renal disease, renal failure, frailty, hearing loss, muscle fatigue, skin conditions, skin wound healing, liver fibrosis, pancreatic fibrosis, oral submucosa fibrosis and sarcopenia; and
(vii) a dermatological disease or disorder is selected from eczema, psoriasis, hyperpigmentation, nevi, rashes, atopic dermatitis, urticaria, diseases and disorders related to photosensitivity or photoaging, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas and cutaneous lupus.

26. A method for treating or preventing a senescence-associated disease or disorder other than a cardiovascular disorder, said method comprising administering to a subject a therapeutically effective amount of an HMG-CoA reductase inhibitor.

27. A method for treating or preventing a senescence-associated disease or disorder, said method comprising administering to a subject a therapeutically effective amount of a focal adhesion kinase (FAK) inhibitor.

28. A method for treating or preventing a senescence-associated disease or disorder by selectively killing senescent cells, said method comprising administering to a subject a therapeutically effective amount of a compound selected from the following: PF-573228, JFD00244, Ouabain, Bufalin, Digoxin, K-Strophanthin, Strophanthidin, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin, Raltegravir, NSC 677249 and Defactinib, and pharmaceutically acceptable salts thereof, where the compound is not already in salt form.

29. Use of a compound selected from the following: PF-573228, JFD00244, Ouabain, Bufalin, Digoxin, K-Strophanthin, Strophanthidin, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin, Raltegravir, NSC 677249 and Defactinib, and pharmaceutically acceptable salts thereof, where the compound is not already in salt form;
in the preparation of a medicament for treating or preventing a senescence-associated disease or disorder, wherein said compound selectively kills one or more senescent cells.

30. Use of a focal adhesion kinase (FAK) inhibitor in the preparation of a medicament for treating or preventing a senescence-associated disease or disorder.

31. Use of an HMG-CoA reductase inhibitor in the preparation of a medicament for treating or preventing a senescence-associated disease or disorder, wherein said senescence-associated disease or disorder is other than a cardiovascular disorder.

32. A senolytic agent which is selected from the following: PF-573228, JFD00244, Ouabain, Bufalin, Digoxin, K-Strophanthin, Strophanthidin, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin, Raltegravir, NSC 677249 and Defactinib, and pharmaceutically acceptable salts thereof, where the compound is not already in salt form.

33. A compound selected from the following: PF-573228, JFD00244, Ouabain, Bufalin, Digoxin, K-Strophanthin, Strophanthidin, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin, Raltegravir, NSC 677249 and Defactinib, and pharmaceutically acceptable salts thereof, where the compound is not already in salt form; for use in extending the lifespan of a subject, preferably by delaying onset or progression of an age-related disease or condition.

34. A compound selected from the following: PF-573228, JFD00244, Ouabain, Bufalin, Digoxin, K-Strophanthin, Strophanthidin, Cyclosporine, Tyrphostin AG879, Cantharidin, Diphenyleneiodonium chloride, Rottlerin, 2,3-Dimethoxy-1,4-naphthoquinone, LY-367,265, Rotenone, Idarubicin, Dequalinium chloride, Vincristine, Atorvastatin calcium, Fluvastatin sodium, Lovastatin, Pitavastatin calcium, Simvastatin, Nitazoxanide, Nitrofurazone, Temsirolimus, Eltrombopag, Adapalene, Azacyclonol, Enoxacin and Raltegravir, and pharmaceutically acceptable salts thereof, where the compound is not already in salt form; for use in treating cancer, and wherein said compound:
reduces or eliminates eliminate cancer cells that have been pushed to senescence; or
reduces or eliminates one or more side effects produced by senescent cells, wherein said side effects comprise inflammation, promotion of cancer growth and promotion of metastasis; or
reduces or eliminates one or more side effects of chemotherapy; or
reduces or eliminates one or more side side effects or radiotherapy; or
reduces or eliminates precancerous lesions; or
reduces or eliminates cells that undergo senescence by treatment with CDK4 or CDK6 inhibitors.
